(19) United States
(12) Patent Application Publication
(10) Pub. No.: US 2024/0247033 A1
(43) Pub. Date: Jul. 25, 2024

(54) SARS-COV-2 RBD CONSTRUCTS

(71) Applicant: The Scripps Research Institute, La Jolla, CA (US)

(72) Inventors: William SCHIEF, Encinitas, CA (US); Jon STEICHEN, San Diego, CA (US); Torben SCHIFFNER, San Diego, CA (US); Xiaozhen HU, San Diego, CA (US); Christoper COTTRELL, Poway, CA (US)

(21) Appl. No.: 18/002,611

(22) PCT Filed: Jun. 22, 2021
(86) PCT No.: PCT/US21/38411

§ 371 Date: Dec. 20, 2022

Related U.S. Application Data
(60) Provisional application No. 63/127,966, filed on Dec. 18, 2020.
(60) Provisional application No. 63/042,435, filed on Jun. 22, 2020.

(51) Int. Cl. C07K 14/005; A61K 39/215; G01N 33/569

(52) U.S. Cl. CPC C07K 14/005; A61K 39/215; G01N 33/56983; C07K 2319/03; C07K 2319/91; C12N 2740/16022; C12N 2770/20022; C12N 2770/20034; G01N 2333/165; G01N 2469/20

(57) ABSTRACT

The present invention relates to glycan-masked and membrane-tethered SARS-CoV-2 RBD vaccine constructs and methods for making and administering the same. The present invention also encompasses a general vaccine platform for coronaviruses.

### RELATED APPLICATIONS AND INCORPORATION BY REFERENCE

This application is claims priority to U.S. provisional patent application Ser. No. 63/042,435 filed Jun. 22, 2020 and U.S. provisional patent application Ser. No. 63/127,966 filed Dec. 18, 2020.

### FEDERAL FUNDING LEGEND

This invention was made with government support under grant number AI1144462 awarded by the National Institutes of Health. The government has certain rights in the invention.

The foregoing applications, and all documents cited therein or during their prosecution (“appln cited documents”) and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to glycan-masked and membrane-tethered SARS-CoV-2 RBD vaccine constructs and a general vaccine platform for coronaviruses.

### BACKGROUND OF THE INVENTION

Coronaviruses (CoVs) have been responsible for several outbreaks over the past two decades, including SARS-CoV in 2002-2003, MERS-CoV in 2012 (de Wit E et al. Nat Rev Microbiol. 2016; 14:523-34), and the current COVID-19 pandemic, caused by SARS-CoV-2, which began in late 2019 (Tse L V et al. Frontiers in microbiology. 2020; 11:658).

COVID-19 has emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. SARS-CoV-2 was isolated and sequenced from human airway epithelial cells from infected patients (Zhu, N. et al. N. Engl. J. Med. 382, 727-733 (2020) and Wu, F. et al. Nature 579, 265-269 (2020)). Disease symptoms range from mild flu-like to severe cases with life-threatening pneumonia (Huang, C. et al. Lancet 395, 497-506 (2020)). The global situation is dynamically evolving, and on 30 Jan. 2020 the World Health Organization declared COVID-19 as a public health emergency of international concern (PHEIC), and on 11 Mar. 2020 it was declared a global pandemic.

Infections have spread to multiple continents. Human-to-human transmission has been observed in multiple countries, and a shortage of disposable personal protective equipment, and prolonged survival times of coronaviruses on inanimate surfaces, have compounded this already delicate situation and heightened the risk of nosocomial infections. The scale of the COVID-19 pandemic has led to unprecedented efforts by the research community to rapidly identify and test therapeutics and vaccines, and to understand the molecular basis of SARS-CoV-2 entry, pathogenesis, and immune targeting.

A sub-region of the SARS-CoV-2 Spike protein, spanning from residue Pro330 to Leu650, possesses a receptor-binding region (RBD) and there is high homology between SARS-CoV-2 and SARS-CoV. Structural studies have identified multiple conformational B cell epitopes and mapped binding of the RBD to the ACE2 receptor (Wrapp et al., Science 367, 1260-1263 (2020) 13 Mar. 2020; Walls et al., Cell 180, 281-292, Apr. 16, 2020). The RBD is predicted to posess B cell (Ser438-Gln506, Thr553-Glu583, Gly404-Aps427, Thr345-Ala352, and Lys529-Lys535) and T cell (9 CD4 and 11 CD8 T cell antigenic determinants) epitopes. (see e.g., Su Q D, et al., The biological characteristics of SARS-CoV-2 spike protein Pro330-Leu650. Vaccine. 2020 Apr. 30:S0264-410X(20)30587-9. doi: 10.1016/j.vaccine.2020.04.070).

In the SARS-CoV, MERS-CoV and SARS-CoV-2 outbreaks, neutralizing antibodies (nAbs) obtained from plasma of recovered patients have been used to decrease viral load and reduce mortality. Instead of polyclonal mixtures, an alternative strategy would be to administer purified monoclonal antibodies with neutralizing capacity there are efforts to identify and produce such antibodies. However, the identification of nAbs from patient serum does not solve the problem of how to make a vaccine that reproducibly elicits a neutralizing antibody response.

Citation or identification of any document in this application is not an admission that such document is available as prior art to the present invention.

### SUMMARY OF THE INVENTION

In an embodiment the invention provides a non-naturally occurring pathogen surface glycoprotein receptor binding domain (RBD) which may comprise an engineered glycosylation site.

In an embodiment the invention provides a non-naturally occurring pathogen surface glycoprotein RBD wherein the engineered glycosylation site may comprise substitution of N at the position to be glycosylated or substitution of S or T at the position two amino acids towards the C-terminus from an existing N of the surface glycoprotein RBD or substitution of N at the position to be glycosylated and substitution of S or T at the position two amino acids towards the C-terminus from the substituted N of the surface glycoprotein RBD, so as to create the motif N-X-S/T, so long as X is not proline.

In an embodiment the invention provides a non-naturally occurring pathogen surface glycoprotein RBD wherein the pathogen is a coronavirus.

In an embodiment the invention provides a non-naturally occurring pathogen surface glycoprotein RBD which may comprise a sequence that is at least 70%, at least 80%, at least 85%, at least 90%, at least 95%, at least 96%, at least 97%, at least 98%, or at least 99%, identical to the RBD of the SARS-CoV-2-S surface glycoprotein; or which may comprise a sequence that is at least 70%, at least 80%, at least 85%, at least 90%, at least 95%, at least 96%, at least 97%, at least 98%, or at least 99%, identical to the RBD of the SARS-CoV-2-S glycoprotein; or which may comprise a sequence as set forth and/or exemplified herein and/or a sequences having at least 95%, 96%, 97%, 98%, or 99% identity to such a sequence as set forth and/or exemplified herein.

In an embodiment the invention provides a non-naturally occurring pathogen surface glycoprotein RBD which may comprise an engineered glycosylation site at one or more of amino acid positions 346, 357, 360, 370, 381, 386, 394, 428, 458, 460, 475, 481, 484, 503, 518, and 522 according to the amino acid numbering of the SARS-CoV-2-S surface glycoprotein.

In an embodiment the invention provides a non-naturally occurring pathogen surface glycoprotein RBD which may comprise, with reference to the amino acid numbering of the SARS-CoV-2-S surface glycoprotein, an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428, or at positions 357, 381, 394, and 518 or at positions 346, 357, 428, 458, 518, 484, and 475 or at positions 346, 357, 428, or 518.

In an embodiment the invention provides a non-naturally occurring pathogen surface glycoprotein RBD which may comprise with reference to the amino acid numbering of the SARS-CoV-2-S surface glycoprotein, an engineered N-glycosylation site at one or two or three or four or five or six or seven or eight of positions 346, 357, 360, 381, 386, 394, 428, 458, 475, 484, 518, and 522, in any combination. In particular embodiments, the pathogen surface glycoprotein RBD may comprise engineered N-glycosylation sites at positions 357, 381, 386, 394, and 428 (SEQ ID NO:13), or at positions 357, 394, 428, 518, and 522 (SEQ ID NO:14), or at positions 357, 394, 428, and 518 (SEQ ID NO:15), or at positions 357, 386, 394, 428, and 518 (SEQ ID NO:16), or at positions 386, 394, 518, and 522 (SEQ ID NO:17), or at positions 357, 381, 386, 394, 428, 518, and 522 (SEQ ID NO:18), or at positions 357, 386, 394, 428, 518, and 522 (SEQ ID NO:19), or at positions 357, 381, 394, and 428 (SEQ ID NO:21), or at positions 357, 381, 394, and 518 (SEQ ID NO:20) or at positions 346, 357, 428, 458, 518, 484, and 475 (SEQ ID NO: 23) or at positions 346, 357, 428, or 518 (SEQ ID NO: 24). In particular embodiments, the pathogen surface glycoprotein RBD may comprise engineered N-glycosylation sites in a combination selected from Table 2.

In certain embodiments, the above pathogen surface glycoprotein RBDs further comprise an N-glycosylation site at one or both of positions 460 and 481, or an N-glycosylation site at one or both of positions 370 and 386.

In an embodiment the invention provides a non-naturally occurring pathogen surface glycoprotein RBD wherein the RBD includes a linker to a transmembrane domain of the pathogen or coronavirus surface glycoprotein for cell surface expression.

In an embodiment the invention provides a non-naturally occurring pathogen surface glycoprotein RBD wherein the linker may comprise a glycine rich linker, or GGSGGSGGSGGSGGS, or a T-cell epitope, or a PADRE CD4 T cell epitope.

In an embodiment the invention provides a non-naturally occurring pathogen surface glycoprotein which may comprise the non-naturally occurring pathogen surface glycoprotein RBD, or a non-naturally occurring coronavirus surface glycoprotein which may comprise the non-naturally occurring pathogen surface glycoprotein RBD as disclosed herein.

In an embodiment the invention provides a non-naturally occurring pathogen or coronavirus surface glycoprotein as disclosed herein including a secretion signal sequence.

In an embodiment the invention provides a non-naturally occurring pathogen or coronavirus surface glycoprotein as disclosed herein including a moiety capable of binding to a metal hydroxide adjuvant; a moiety capable of binding to a metal hydroxide adjuvant at or near which may comprise within 25 amino acids of the N- or C-terminus; a moiety capable of binding to a metal hydroxide adjuvant which may comprise phosphoserine; a moiety capable of binding to a metal hydroxide adjuvant at or near which may comprise within 25 amino acids of the N- or C-terminus which may comprise phosphoserine; a moiety capable of binding to a metal hydroxide adjuvant which may comprise cysteine; a moiety capable of binding to a metal hydroxide adjuvant at or near which may comprise within 25 amino acids of the N- or C-terminus which may comprise cysteine; or any of the foregoing wherein the metal hydroxide adjuvant may comprise aluminum hydroxide or alum or sodium bis(2-methoxyethoxy)aluminum hydride; or any of the foregoing which may comprise phosphoserine that can couple with a cysteine.

In an embodiment the invention provides a non-naturally occurring nucleic acid molecule encoding the non-naturally occurring pathogen surface glycoprotein RBD as disclosed herein or the non-naturally occurring pathogen or coronavirus surface glycoprotein as disclosed herein.

In an embodiment the invention provides a vector which may comprise a regulatory element operable in a eukaryotic cell operably linked to a nucleic acid disclosed herein.

In an embodiment the invention provides a vector as disclosed herein wherein the vector may comprise a DNA or DNA plasmid vector.

In an embodiment the invention provides a vector as disclosed herein wherein the vector may comprise an RNA or mRNA vector.

In an embodiment the invention provides a vector as disclosed herein wherein the vector may comprise a cellular eukaryotic organism, a eukaryotic cell, a mammalian cell, a 293 cell, a VERO cell, a CHO (Chinese Hamster Ovary) cell, a viral vector or a yeast.

In an embodiment the invention provides a vector as disclosed herein which may comprise the eukaryotic cell, the mammalian cell, the 293 cell, the VERO cell, or the CHO cell.

In an embodiment the invention provides a vector as disclosed herein which may comprise a viral vector.

In an embodiment the invention provides a vector as disclosed herein wherein the viral vector may comprise a DNA virus, a RNA virus, a replicon RNA virus, an alphavirus, a flavivirus, a measles virus, a rhabdovirus, a baculovirus, a poxvirus, a vaccinia virus, an avipox virus, a canarypox a fowlpox virus, a dovepox virus, a modified vaccinia Ankara (MVA), a NYVAC vaccinia virus, an ALVAC canarypox virus, a TROVAC fowlpox virus, an MVA-BN, a herpesvirus, an adenovirus, an adeno-associated virus (AAV), a vesicular stomatitis virus (VSV), a chimeric virus expressing as a surface protein the non-naturally occurring pathogen or coronavirus surface glycoprotein or the non-naturally occurring pathogen surface glycoprotein RBD.

In an embodiment the invention provides an immunogenic or vaccine composition which may comprise a pharmaceutically or veterinarily acceptable carrier and an effective amount to elicit an immune response, or an effective amount to elicit a protective immune response, of: the non-naturally occurring pathogen surface glycoprotein RBD, or the non-naturally occurring pathogen or coronavirus surface glycoprotein, or the non-naturally nucleic acid molecule, or the vector, as disclosed herein.

In an embodiment, the vaccine or immunogenic composition may comprise or can be a subunit, DNA, DNA plasmid, mRNA, inactivated live chimeric (for example, but not limited to, chemical or ultraviolet inactivation), live chimeric; lyophilized; lyophilized and a constituent, readily water dissolveable, dispersing e.g. effervescent on admixing with water, in powder form, in tablet form, in liquid form, or aerosolized.

In an embodiment, the vaccine or immunogenic composition is or can be administered, without limitation, orally, nasally, perilingually, sublingually, rectally, subcutaneously, intradermally, or by injection.

In an embodiment, the vaccine or composition is or can be administered alone, as a single as a single administration; or administered as part of immunization/vaccination regimen such as bi-annually, annually, once or twice or thrice or quarterly or more such as monthly and/or a prime-boost regimen including where prime and boost same or different presentations of antigen (surface glycoprotein).

In an embodiment, the vaccine or composition is or can be administered in a regimen wherein regimen may comprise administration of one or more immunogenic or vaccine composition against another pathogen, e.g. influenza such as a coronavirus and influenza vaccination or immunization regimen.

In an embodiment, the vaccine or composition is or can be administered as part of a combination vaccine or co-administration or sequential administration with an immunogenic or vaccine composition against another pathogen, e.g. a combination of a coronavirus and influenza.

In an embodiment the invention provides a vaccine or immunogenic composition, including an adjuvant.

In an embodiment the invention provides in the composition including an adjuvant, the adjuvant may comprise aluminum hydroxide, or alum, or sodium bis(2-methoxyethoxy)aluminum hydride, or an oil-in-water adjuvant, water-in-oil adjuvant, or a carbomer adjuvant.

In an embodiment the invention provides a vaccine or immunogenic composition wherein the composition may comprise the non-naturally occurring pathogen surface glycoprotein RBD, or the non-naturally occurring pathogen or coronavirus surface glycoprotein, and the adjuvant may comprise aluminum hydroxide, or alum, or sodium bis(2-methoxyethoxy)aluminum hydride.

In an embodiment the invention provides a vaccine or immunogenic composition which may comprise the non-naturally occurring pathogen or coronavirus surface glycoprotein which may comprise the moiety capable of binding to a metal hydroxide adjuvant; or the moiety capable of binding to a metal hydroxide adjuvant at or near which may comprise within 25 amino acids of the N- or C-terminus; a moiety capable of binding to a metal hydroxide adjuvant which may comprise phosphoserine; or the moiety capable of binding to a metal hydroxide adjuvant at or near which may comprise within 25 amino acids of the N- or C-terminus which may comprise phosphoserine; or the moiety capable of binding to a metal hydroxide adjuvant which may comprise cysteine; or the moiety capable of binding to a metal hydroxide adjuvant at or near which may comprise within 25 amino acids of the N- or C-terminus which may comprise cysteine; wherein the adjuvant couples with the pathogen or coronavirus surface glycoprotein.

In an embodiment the invention provides a method for producing the non-naturally occurring pathogen surface glycoprotein RBD, or the non-naturally occurring pathogen or coronavirus surface glycoprotein as disclosed herein which may comprise expressing a non-naturally nucleic acid molecule as disclosed herein, or expressing a non-naturally nucleic acid molecule from a vector as disclosed herein; and optionally recovering, isolating and/or purifying the non-naturally occurring pathogen surface glycoprotein RBD, or the non-naturally occurring pathogen or coronavirus surface glycoprotein. Advantageously, for a subunit vaccine there is the recovering, isolating and/or purifying.

In an embodiment the invention provides a method for eliciting an immune or protective immune response in a mammal, or for eliciting, stimulating or producing an antibody or antibody response in a mammal, or for eliciting, stimulating or producing a neutralizing antibody (nAb) response in a mammal which may comprise administering an effective amount of the non-naturally occurring pathogen surface glycoprotein RBD, or the non-naturally occurring pathogen or coronavirus surface glycoprotein as disclosed herein or a vaccine or immunogenic composition as disclosed herein; or, expressing in vivo a non-naturally occurring nucleic acid molecule herein disclosed, or, expressing in vivo a non-naturally nucleic acid molecule herein disclosed from a vector herein disclosed. In an embodiment the invention provides such a method wherein the mammal is a human, a non-human primate, a rodent, a chiroptera, or a bat, or a canine, or a dog, or a feline, or a cat, or a porcine, or a pig, or an equine, or a horse, or a bovine, or a cow or bull, or a mammal that may comprise elements of a human immune system. In an embodiment the invention provides such methods of the foregoing sentences of this paragraph wherein the mammal is capable of producing human antibodies.

In an embodiment the invention provides a non-naturally occurring pathogen surface glycoprotein RBD, or the non-naturally occurring pathogen or coronavirus surface glycoprotein contains the linker and/or the secretion signal sequence as herein above discussed.

In an embodiment the invention provides a non-naturally occurring coronavirus surface glycoprotein receptor binding domain (RBD) which may comprise an engineered glycosylation site at one or more of amino acid positions 346, 357, 381, 386, 394, 428, 458, 475, 484 and 518 according to the amino acid numbering of the SARS-CoV-2-S surface glycoprotein. In an embodiment the invention provides a non-naturally such a RBD wherein the RBD may comprise a sequence that is at least 70%, at least 80%, at least 85%, at least 90%, at least 95%, at least 96%, at least 97%, at least 98%, or at least 99%, identical to the RBD of the SARS-CoV-2-S surface glycoprotein. In an embodiment the invention provides a non-naturally RBD as discussed in this paragraph, wherein the RBD may comprise the RBD of the SARS-CoV-2-S surface glycoprotein. In an embodiment the invention provides a non-naturally RBD as discussed in this paragraph, wherein the RBD may comprise a sequence that is at least 70%, at least 80%, at least 85%, at least 90%, at least 95%, at least 96%, at least 97%, at least 98%, or at least 99%, identical to the RBD of the SARS-CoV-S surface glycoprotein. In an embodiment the invention provides a non-naturally RBD as discussed in this paragraph, wherein the RBD, wherein the RBD may comprise the RBD of the SARS-CoV-S surface glycoprotein. (SARS-CoV-2-S understood to be on the surface of COVID-19; SARS-CoV-S understood to be on the surface of the 2002 SARS virus.) An engineered glycosylation site may comprise substitution of “N” at the position intended to be glycosylated or substitution of S or T two amino acids towards the C-terminal from a pre-existing “N” intended to be glycosylated. The consensus sequence is Asn-X-Ser/Thr where X is any amino acid except proline. In an embodiment the invention provides a non-naturally RBD as discussed in this paragraph, wherein the RBD may comprise an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428. In an embodiment the invention provides a coronavirus surface glycoprotein, which may comprise a RBD of any aspect of this paragraph. In an embodiment the invention provides a hybrid protein which may comprise a RBD of any aspect of this paragraph. In an embodiment the invention provides such a hybrid protein which may comprise the RBD operatively linked to a transmembrane domain and/or a secretion signal sequence. In an embodiment of the invention, the hybrid protein can have the RBD is operatively linked to the transmembrane domain by a linker or flexible linker, such as a G rich linker or flexible linker or a linker including a T cell epitope such as a PADRE CD4 T cell epitope.

In an embodiment the invention provides the RBD, or the pathogen or coronavirus surface glycoprotein or the hybrid protein operatively linked to a T cell epitope, such as a PADRE CD4 T cell epitope.

In an embodiment, the invention provides a conjugate which may comprise the RBD, coronavirus surface glycoprotein, or hybrid protein as discussed herein and a moiety capable of binding to a metal hydroxide adjuvant.

In an embodiment, the invention provides a pharmaceutical composition which may comprise the RBD, coronavirus surface glycoprotein, or hybrid protein, or conjugate as herein disclosed.

In an embodiment, the invention provides an isolated and/or non-naturally occurring nucleic acid encoding any herein disclosed molecule. In an embodiment, the invention provides a vector which may comprise a regulatory element operable in a eukaryotic cell operably linked to the nucleic acid molecule. The vector can be any vector as herein discussed, including that the vector can comprise a viral vector, such as AAV, VSV, or a chimeric vector (e.g., VSV or another virus expressing the RBD or surface glycoprotein of the invention on the surface of the virus).

In an embodiment the invention provides methods of eliciting an immune response in a mammal which may comprise administering a molecule as herein disclosed. The method can result in stimulating a neutralizing antibody (nAb) in the mammal by the method which may comprise administering the molecule. The mammal can be any mammal herein discussed, such as a human or a non-human primate, or a mammal having elements of a human immune system, or a mammal is capable of producing human antibodies. In an embodiment the method includes administering with an adjuvant, such as, for example, alum.

In another aspect, the invention provides a diagnostic serological probe. In an embodiment of the invention, one or more epitopes that bind to a non-nAb is masked, providing a probe capable of detecting one or more nAbs in serum or other antibody mixture. In another embodiment, one or more epitopes that bind to a nAb is masked, providing a probe capable of detecting one or more non-nAbs in serum or other polyclonal mixture.

In another aspect, the invention provides a diagnostic serological probe tailored to reduce receptor binding. As demonstrated herein, adding a glycosylation site at N503 of SARS-CoV-2 reduced ACE binding to undetectable levels while binding to other nAbs and non-nAbs was diminished by at most, less that a factor of 10.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “which may comprise” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A presents an amino acid sequence for mem_RBD_v058 (referred to in FIG. 1A as SARS2_RBD_v058). This construct includes five engineered glycosylation sites, at positions N357, N381, N386, N394, N428, and it includes a GGS linker between the RBD and TM domains. This construct expresses well on the cell surface and has an excellent antigenic profile in which neutralizing antibodies bind but non-neutralizing or weakly-neutralizing antibodies show no detectable binding.

FIG. 1B presents an amino acid sequence for mem_RBD_v059 (referred to in FIG. 1A as SARS2_RBD_v059). This construct includes five engineered glycosylation sites, at positions N357, N381, N386, N394, N428, and includes a PADRE linker between RBD and TM domains. This construct is the same as SEQ 1 except it uses a PADRE linker instead of a GGS linker. This construct also has an excellent antigenic profile.

FIG. 2 depicts a schematic illustrating the base design for the memRBD vaccine constructs. Glycosylation sites are added to surfaces on RBD that would be occluded on the SARS-CoV-2 Spike, for the purpose of inhibiting the binding or induction of non-neutralizing antibodies targeting such surfaces. Exogeneous CD4 T help can be added to these constructs, as exemplified by the addition of a PADRE epitope to the tether in the v059 construct. In the examples shown, Applicants have used a TM domain from HIV BG505 that they have separately verified has good expression, but other TM domains would likely suffice.

FIG. 3 depicts the structure of Sars-CoV-2_S protein in 1 RBD up conformation showing the location of glycosylation sites (red spheres) that were tested in the mammalian display library. In order to determine the location of ACE2 binding, the structure of ACE2 in complex with the RBD was aligned to the RBD in the trimer structure. ACE2 is colored cyan, RBD is yellow, Spike protein is gray. These added glycans were also positioned so as not to interfere with ACE2 receptor binding.

FIG. 4 depicts cell-surface antigenic profiles of SARS-CoV-2 2P-stabilized spike, two memRBD constructs, and a negative control protein. Freestyle 293-F cells were transfected with the indicated DNAs according to manufacturer's instructions (ThermoFisher Scientific). Two days after transfection, cells were stained with the indicated antibodies or recombinant Fc-tagged human ACE2 (hACE2-Fc) at 1 μg/mL in FACS buffer (PBS+0.1% BSA), washed twice, labelled with Alexa Fluor 647-conjugated anti-human IgG (Jackson Immunoresearch) and analyzed on a a NovoCyte 3000 (ACEA Biosciences). Data were analyzed by gating on single cells using FSC, SSC and width in FlowJo 10.6.2 (Beckton Dickinson). The most frequent fluorescence intensity of the Alexa-647 signal was calculated and plotted in Prism 8 for macOS (Graphpad). Neutralizing and nonneutralizing antibodies are from Rogers et al. Science 2020 (DOI: 10.1126/science.abc7520). Some antibodies described here as “non-nAbs” do have neutralizing activity but are very weak. Both memRBD constructs show qualitatively “perfect” antigenicity for nAb elicitation: binding to nAbs but not to non-nAbs. Thus, memRBD constructs may preferentially elicit nAbs rather than non-nAbs. In contrast, the SARS-CoV-2 2P-stabilized Spike (SARS2-S-2P) binds both nAbs and non-nAbs, thus SARS2-S-2P is likely to induce both types of Abs. MemRBD_v058 shows similar ACE2 binding as SARS2-S-2P, but substantially higher binding to human nAbs, likely reflecting higher expression level on the cell surface. Increased expression levels would allow for further dose sparing compared to full-length spike.

FIG. 5 depicts SPR dissociation constants for glycan-masked RBDs binding to ACE2, nAbs, and non-nAbs, in which the glycan masking has caused substantial (10-fold or more) reduction in affinity (increase in K_{D}) for ACE2 or one or more nAbs.

FIG. 6 depicts SPR dissociation constants for glycan-masked RBDs binding to ACE2, nAbs, and non-nAbs, in which the glycan masking has caused substantial (10-fold or more) reduction in affinity (increase in K_{D}) for non-nAbs without causing substantial reduction in affinity for ACE2 or one or more nAbs.

FIG. 7 depicts a structural model of RBD (cyan) binding to an ACE2 monomer (green), with positions of candidate glycosylation sites on RBD indicated by spheres. Blue spheres indicate sites 460 and 481; the red sphere indicates site 370; and cyan spheres indicate the sites tested by mammalian display: 357, 360, 381, 394, 428, 518, 522.

FIG. 8 depicts cell-surface antigenic screening of additional memRBD variants g3.6 (SEQ ID NO:20) and g3.7 (SEQ ID NO:21). 293F-cells transfected with the indicated DNA constructs were stained with RBD-specific mAbs at 1 μg/mL, labelled with Alexa Fluor 647-conjugated anti-human IgG and analyzed by flow cytometry. The RBD-specific potent neutralizing antibody CC6.30 was used as a readout for overall RBD expression levels. Antibodies CC12.19 and CR3022 were used to test binding of undesired weakly or non-neutralizing antibodies targeting the RBD-B and RBD-C sites, respectively. Both CC12.19 and CR3022 bound strongly to constructs with wildtype RBDs (WT-RBD). By contrast, all variants that incorporated glycans to mask these epitopes efficiently suppressed binding of both CC12.19 and CR3022. This demonstrates the importance of the glycan masking. The data also showed that increasing the length of the GGS-linker between the RBD and the trans-membrane domain increases overall expression. Incorporating a PADRE-epitope into the linker also led to strong expression, although it was lower than for the longest GGS-linker tested. Adding the PADRE epitope to the intracellular c-terminus of the protein had only minor effects on expression levels compared to the matched construct with the same linker length. Expression levels of g3.6 were comparable to WT-RBD, whereas most g3.7 constructs showed lower expression. memRBD_v058 and memRBD_v059, which add an additional glycan to g3.7, had further reduced expression levels. These data support the utility especially of the g3.6 design, as it displays higher expression than v058/v059 but similarly favorable antigenic profile.

FIG. 9 depicts locations of glycan masking sites on SARS-CoV-2 RBD. Locations of glycan masking sites on SARS-CoV-2 RBD. Positions are indicated for engineered glycosylation sites for masking the RBS and sites outside the RBS. The SARS-CoV-2 RBD is shown in surface representation in four different orientations, with each panel differing from its neighbors by 900 rotation about a vertical axis. Surface coloring as follows: RBS, green; RBS masking glycosylation sites, purple; glycosylation sites masking non-RBS epitopes, blue.

FIG. 10 depicts glycan masking of non-conserved or non-neutralizing epitopes in SARS-CoV-2 RBD, including masking of the non-conserved immunodominant receptor binding site (RBS). Dissociation constants measured by SPR for hACE2 and the indicated antibodies binding to wild-type (WT) and engineered receptor binding domains (RBDs). SARS2-WT, wild-type RBD for SARS-CoV-2; SARS1-WT, wild-type RBD for SARS-CoV; SARS2 v183, glycan-masked RBD for SARS-CoV-2 with glycans added at the indicated positions to mask non-conserved or non-neutralizing sites outside the RBS; SARS2 v178, glycan-masked RBD for SARS-CoV-2 with glycans added at the indicated positions to mask the RBS and to mask non-conserved or non-neutralizing sites outside the RBS. The data show that glycan masking eliminates or weakens binding of hACE2 and RBS-specific antibodies but maintains high-affinity binding to cross-neutralizing antibodies. The key shows epitope classification obtained from published literature as: “RBS-X” from M. Yuan et al., Science 10.1126/science.abh1139 (2021) and “Class X” from Barnes et al., Nature 2020 https://doi.org/10.1038/s41586-020-2852-1.

FIG. 11 depicts SARS-CoV-2 RBD with 0 glycosylation sites added (top) and SARS-CoV-2 RBD with 7 glycosylation sites added: 3 to mask RBS (458, 475, 484) and 4 to mask non-conserved or non-neutralizing epitopes (346+357+428+518) (bottom). ELISA measurement of antibody binding to WT SARS-CoV-2 RBD and glycan-masked SARS-CoV-2 RBD. RBDs were captured on the ELISA plate. hACE2-Fc or the indicated IgGs were titrated for binding, and the area under the response curve (AUC) was calculated in Prism. SARS_CoV2_RBD, wild-type RBD for SARS-CoV-2; SARS2_RBD_v178, glycan-masked RBD for SARS-CoV-2 with glycans added to mask the RBS and to mask non-conserved or non-neutralizing sites outside the RBS. The data show that glycan masking effectively eliminates or greatly weakens binding of hACE2 and RBS-specific antibodies but maintains high avidity binding to cross-neutralizing antibodies.

### DETAILED DESCRIPTION OF THE INVENTION

The present invention relates to non-naturally occurring proteins, in particularly glycan masked and/or membrane-tethered SARS-CoV-2 receptor binding domain (RBD) constructs (memRBD vaccine constructs) which may be involved in eliciting an immune response against SARS-CoV-2.

The constructs of the present invention elicit focused neutralizing antibody responses, in contrast to the full length spike, which induces both neutralizing antibody and non-neutralizing antibody responses. This allow for dose sparing and avoid problems associated with non-neutralizing antibody elicitation. In some cases, the cell-surface expression level is higher than for the full length spike, which also allow for dose sparing. The construct is smaller than the full length spike, thus further allowing for dose sparing in the case of nucleic acid delivery and for easier incorporation into viral vectors.

Several recent papers describe neutralizing (nAbs) and non-neutralizing antibodies from convalescent donors. Several studies isolate nAbs (range of potencies, from highly potent to very weak) and non-nAbs, from convalescent donors. The majority of potent nAbs bind RBD and compete with ACE2. The most potent nAbs compete with ACE2 and protect against challenge in animal models. (T. F. Rogers et al., Science 10.1126/science.abc7520 (2020), P. J. M. Brouwer et al., Science 10.1126/science.abc5902 (2020), A. Z. Wec et al., Science 10.1126/science.abc7424 (2020), J. Hansen et al., Science 10.1126/science.abd0827 (2020), Wu et al., Science 368, 1274-1278 (2020), Ju, B. et al., Nature https://doi.org/10.1038/s41586-020-2380-z (2020), Seydoux et al. https://doi.org/10.1101/2020.05.12.091298doi: bioRxiv preprint, and Robbiani et al. https://doi.org/10.1101/2020.05.13.092619doi: bioRxiv preprint). Seydoux et al. indicates that the most potent nAb is called CV30 and the structure is reported by Hurlburt et al. (https://doi.org/10.1101/2020.06.12.148692doi: bioRxiv preprint). The structure of CV30 shows that the nAb binds to RBD with the epitope overlapping that of ACE2.

Structural studies of spike and/or RBD with or without antibodies or ACE2 have also been performed. Yan et al. (Science 367, 1444-1448 (2020)) reports the structure of ACE2 binding to RBD. Walls et al (2020, Cell 180, 281-292) reports the structure and antigenicity of the SARS-CoV-2 spike protein (the entire trimer). Wrapp et al (Science 367, 1260-1263 (2020)) reports the structure and antigenicity of the SARS-CoV-2 spike protein (the entire trimer).

The main target for nAbs on coronaviruses is the spike (S) protein that is anchored in the viral membrane. While epitopes capable of eliciting neutralizing antibodies must exist, the epitopes may be hidden and/or be insufficiently immunodominant to reliably induce a neutralizing antibody response. The S protein comprises two subdomains, the N-terminal S1 domain, which contains the N-terminal domain (NTD) and the receptor-binding domain (RBD) and the S2 domain. Upon receptor binding and membrane fusion, the S protein undergoes a conformational change from a prefusion state to a postfusion state compatible with merging of viral and target cell membranes. While most nAb epitopes may be presented on the prefusion conformation, when expressed as recombinant proteins, S proteins have a propensity to switch to the postfusion state.

The invention provides SARS antigens designed to promote induction of nAbs against SARS-CoV-2. The S protein of SARS-CoV-2 and SARS-CoV show considerable structural and sequence homology. Accordingly, the invention further provides SARS antigens that promote induction of nAbs against SARS-CoV and related viruses.

The invention also encompasses additional glycans and refined glycan positioning, enhanced CD4 T help, including on the intracellular side of the TM domain, multimerization to increase B cell activation and alternate transmembrane domains for further improved expression.

Rogers confirmed existence of neutralizing antibodies which help to define protective epitopes and guide vaccine design in humans. (Rogers et al., Isolation of potent SARS-CoV-2 neutralizing antibodies and protection from disease in a small animal model. Science 15 Jun. 2020: eabc7520, DOI: 10.1126/science.abc7520). The glycoprotein RBDs disclosed herein are effective to induce neutralizing antibodies accordingly.

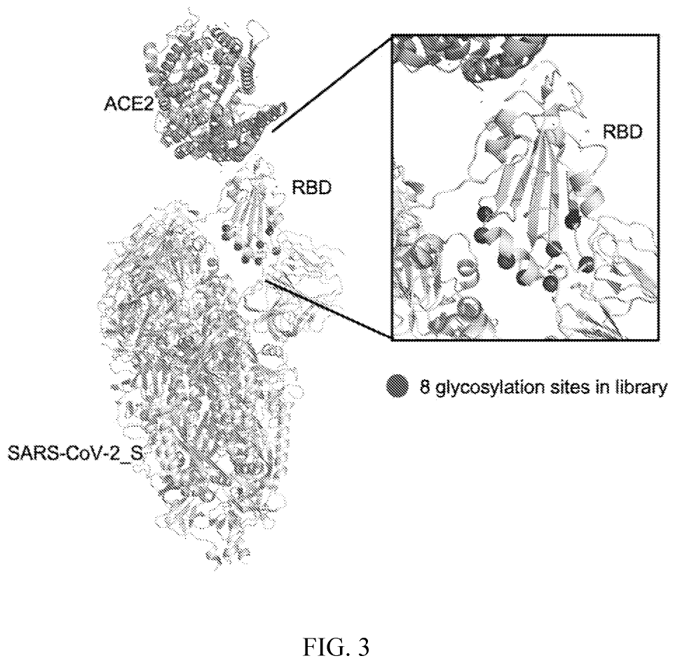

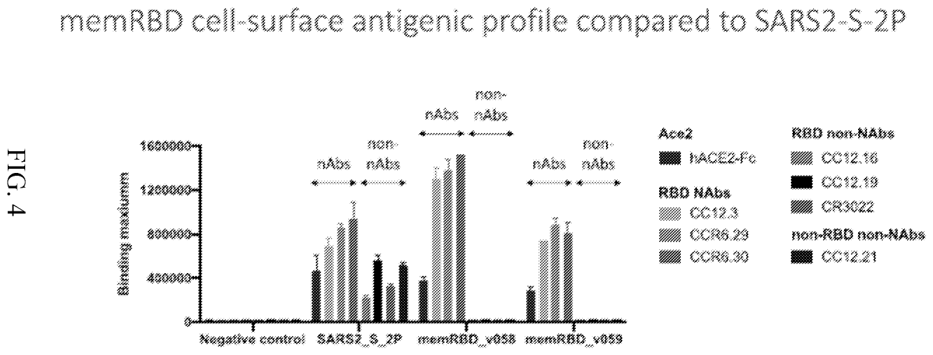

Applicants have designed immunogens that aim to elicit potent neutralizing antibodies against the receptor binding domain (RBD) of SARS-CoV-2. The immunogens are based on the RBD tethered to a transmembrane domain via a flexible linker (FIG. 2). Glycosylation sites have been engineered into the RBD in order to mask the portion of the RBD surface that would be occluded on the SARS-CoV-2 spike trimer (FIGS. 2 and 3). Antibodies targeting surfaces occluded on the trimer should be non-neutralizing. The purpose of the engineered glycosylation sites is to prevent binding or elicitation of non-neutralizing antibodies. The added glycans block binding of non-neutralizing or weak-neutralizing RBD antibodies but do not hinder binding of potent neutralizing RBD antibodies (FIG. 4), thus these vaccine constructs elicit a focused, potently neutralizing response. This focused response allows for protective responses from lower vaccine doses, reducing the cost of each dose and increasing the number of people that can be vaccinated from one batch of vaccine. The tether optionally includes a universal Pan DR epitope (PADRE) CD4 T cell epitope, which increase B cell responses in diverse humans (FIG. 2). Delivery of such constructs would be by nucleic acid or viral vector approaches. The small size of the memRBD construct compared to the full-length spike protein would provide other advantages: it would further contribute to dose sparing for nucleic acid delivery, and, in the context of viral vector delivery the smaller size of the insert would reduce the burden on viral fitness.

N-linked glycosylation involves attachment of a carbohydrate consisting of several sugar molecules, sometimes also referred to as glycan, to the amide nitrogen of an asparagine (Asn) residue of a protein. This type of linkage is important for both the structure and function of many eukaryotic proteins. The N-linked glycosylation process occurs in eukaryotes and widely in archaea, but very rarely in bacteria. The nature of N-linked glycans attached to a glycoprotein is determined by the protein, and the cell in which it is expressed, and varies across species. The carbohydrate consists of sugar moieties, linked to one another in via glycosidic bonds. Attachment of a glycan residue to a protein requires the presence of the consensus sequence Asn-X-Ser/Thr wherein X is any amino acid except proline (Pro). Different species synthesize different types of N-linked glycan.

In an advantageous embodiment, the invention relates to a non-naturally occurring protein which may comprise any one of:

SEQ 1: mem_RBD_v058
MGILPSPGMPALLSLVSLLSVLLMGCVAETGTNLCPFGEVENATR
FASVYAWNRKNISNCVADYSVLYNSASFSTFKCYNVSPTNLTDLC
FTNVSADSFVIRGDEVRQIAPGQTGKIADYNYKLPDNFTGCVIAW
NSNNLDSKVGGNYNYLYRLFRKSNLKPFERDISTEIYQAGSTPCN
GVEGFNCYFPLQSYGFQPTNGVGYQPYRVVVLSFELLHAPATVCG
PGGSGGSGGSGGSGGSGGSKIFIMIVGGLIGLRIVFAVLSVIHR
VR**

SEQ ID NO:1 includes five engineered glycosylation sites, at positions N357, N381, N386, N394, N428, and it includes a GGS linker (SEQ ID NO:3) between the RBD and TM domains. This construct expresses well on cell surface and has excellent antigenic profile in which neutralizing antibodies bind but non-neutralizing or weakly-neutralizing antibodies show no detectable binding, as shown in FIG. 4.

SEQ 2: memRBD_v059
MGILPSPGMPALLSLVSLLSVLLMGCVAETGTNLCPFGEVFNATR
FASVYAWNRKNISNCVADYSVLYNSASFSTFKCYNVSPTNLTDLC
FTNVSADSFVIRGDEVRQIAPGQTGKIADYNYKLPDNFTGCVIAW
NSNNLDSKVGGNYNYLYRLFRKSNLKPFERDISTEIYQAGSTPCN
GVEGFNCYFPLQSYGFQPTNGVGYQPYRVVVLSFELLHAPATVCG
PGGSAKFVAAWTLKAAAGGSKIFIMIVGGLIGLRIVFAVLSVIHR
VR**

SEQ ID NO:2 includes five engineered glycosylation sites, at positions N357, N381, N386, N394, N428, and includes a PADRE linker (SEQ ID NO:4) between RBD and TM domains. SEQ ID NO: 2 is the same as SEQ ID NO: 1 except it uses a PADRE linker instead of a GGS linker. This construct also has an excellent antigenic profile, as shown in FIG. 4.

A secretion signal sequence from the pHLsec vector (MGILPSPGMPALLSLVSLLSVLLMGCVAETG; SEQ ID NO:5) is indicated above, but any secretion signal sequence may be contemplated. The leader sequence will be cleaved during expression/secretion and is not present in the final expressed protein product. The embodiments contained herein are not limited to this particular leader sequence as different leader sequences could be used to serve the same purpose.

The transmembrane domain (TM) from HIV Env of the BG505 isolate (KIFIMIVGGLIGLRIVFAVLSVIHRVR; SEQ ID NO:6), but any TM domain will suffice. As examples, other TM domains could include the TM from SARS-CoV-2 (KWPWYIWLGFIAGLIAIVMVTIML; SEQ ID NO:7) or the TM from VSV-G (KSSIASFFFIIGLIIGLFLVLR; SEQ ID NO:8).

Other glycosylation sites may be added to mask additional parts of the RBD. The most potent SARS-CoV-2 neutralizing antibodies are directed to the ACE2-binding site, so additional glycans may be added to mask all except the ACE2-binding site.

The consensus sequence for N-glycosylation is Asn-Xaa-Ser/Thr wherein Xaa can be any amino acid except proline. Thus, one way to introduce an N-glycosylation site is by substituting in an Asn residue two amino acids N-terminal to a Ser or Thr residue. Another way to introduce an N-glycosylation site is by substituting in a Ser or Thr residue two amino acids downstream from a preexisting Asn residue. A third way to introduce an N-glycosylation site is by substituting an Asn residue at a desired location and a Ser or Thr residue two amino acids towards the C terminal of the protein.

The exemplary SARS-CoV-2 proteins disclosed herein comprise N-glycosylation sites introduced at one or more of amino acid positions 346, 357, 360, 370, 381, 386, 428, 458, 460, 475, 481, 484, 503, 518, and 522 by substituting amino acid residues in the SARS-CoV-2 RBD (SEQ ID NO:9) as follows: aa 357: R357N; aa 360: V362S OR V362T; aa 370: A372S or A372T; aa 381: G381N; aa 386: K386N with N388T or N388S; aa 394: Y396S or Y396T; aa 428: D428N; aa 460: K462S or K462T; aa 481: V483S or V483T; aa 503: V503N with Y505S or Y505T; aa 518: L518N with A520S or A520T; aa 522: A522N with V524S or V524T. Likewise, N-glycosylation sites can be added at corresponding positions of other coronaviruses, including but not limited to SARS-CoV, MERS-CoV, and mutants of SARS-CoV-2, SARS-CoV, and MERS-CoV as they exist or arise in the population from time to time.

In certain embodiments, the invention provides coronavirus RBDs and surface glycoproteins which may comprise, with reference to the amino acid numbering of the SARS-CoV-2-S surface glycoprotein, an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428.

In certain embodiments, the invention provides coronavirus RBDs and surface glycoproteins which may comprise, with reference to the amino acid numbering of the SARS-CoV-2-S surface glycoprotein, an engineered glycosylation site at an engineered N-glycosylation site at one or two or three or four or five or six or seven or eight of positions 357, 360, 381, 386, 394, 428, 518, and 522, in any combination. In particular embodiments, the invention provides coronavirus RBDs and surface glycoproteins which may comprise an engineered N-glycosylation site at positions 357, 381, 386, 394, and 528, or at positions 357, 394, 428, 518, and 522, or at positions 357, 394, 428, and 518, or at positions 357, 386, 394, 428, and 518, or at positions 386, 394, 518, and 522, or at positions 357, 381, 386, 394, 428, 518, and 522, or at positions 357, 386, 394, 428, 518, and 522. In particular embodiments, the invention provides coronavirus RBDs and surface glycoproteins which may comprise engineered N-glycosylation sites in a combination selected from Table 2.

In certain of the above embodiments, the invention further provides N-glycosylation site at one or both of positions 460 and 481, or an N-glycosylation site at one or both of positions 370 and 386.

The following table provides amino acid sequences of the basic components discussed above, including the SARS-CoV-2 RBD and surface glycoprotein amino acid sequences used for reference.

Table of Sequences

| SEQ | | |
|---|---|---|
| ID | Protein or | |
| NO | Component | Amino Acid Sequence |
| 1 | memRBD_v058 | MGILPSPGMP ALLSLVSLLS VLLMGCVAET GTNLCPFGEV FNATRFASVY |
| | | AWNRKNISNC VADYSVLYNS ASFSTFKCYN VSPTNLTDLC FTNVSADSFV |
| | | IRGDEVRQIA PGQTGKIADY NYKLPDNFTG CVIAWNSNNL DSKVGGNYNY |
| | | LYRLERKSNL KPFERDISTE IYQAGSTPCN GVEGENCYFP LQSYGFQPTN |
| | | GVGYQPYRVV VLSFELLHAP ATVCGPGGSG GSGGSGGSGG SGGSKIFIMI |
| | | VGGLIGLRIV FAVLSVIHRV R |
| 2 | memRBD_v059 | MGILPSPGMP ALLSLVSLLS VLLMGCVAET GTNLCPFGEV FNATRFASVY |
| | | AWNRKNISNC VADYSVLYNS ASFSTFKCYN VSPTNLTDLC FTNVSADSFV |
| | | IRGDEVRQIA PGQTGKIADY NYKLPDNFTG CVIAWNSNNL DSKVGGNYNY |
| | | LYRLFRKSNL KPFERDISTE IYQAGSTPCN GVEGFNCYFP LQSYGFQPTN |
| | | GVGYQPYRVV VLSFELLHAP ATVCGPGGSA KEVAAWTLKA AAGGSKIFIM |
| | | IVGGLIGLRI VFAVLSVIHR VR |
| 3 | GGS linker | GGSGGSGGSG GSGGSGGS |
| 4 | PADRE linker | GGSAKFVAAW TLKAAAGGS |
| 5 | pHLsec | MGILPSPGMP ALLSLVSLLS VLLMGCVAET G |
| 6 | HIV env TM | KIFIMIVGGL IGLRIVFAVL SVIHRVR |
| 7 | SARS-CoV-2 TM | KWPWYIWLGF IAGLIAIVMV TIML |
| 8 | VSV-G TM | KSSIASFFFI IGLIIGLFLV LR |
| 9 | SARS-CoV-2 | TNLCPFGEVF NATRFASVYA WNRKRISNCV ADYSVLYNSA SFSTFKCYGV |
| | RBD | SPTKLNDLCF TNVYADSFVI RGDEVRQIAP GQTGKIADYN YKLPDDFTGC |
| | GenBank | VIAWNSNNLD SKVGGNYNYL YRLFRKSNLK PFERDISTEI YQAGSTPCNG |
| | QHD43416.1 | VEGFNCYFPL QSYGFQPTNG VGYQPYRVVV LSFELLHAPA TVCGP |
| 10 | SARS-CoV | TNLCPFGEVF NATKFPSVYA WERKKISNCV ADYSVLYNST FFSTFKCYGV |
| | RBD | SATKLNDLCF SNVYADSFVV KGDDVRQIAP GQTGVIADYN YKLPDDFMGC |
| | GenBank | VLAWNTRNID ATSTGNYNYK YRYLRHGKLR PFERDISNVP FSPDGKPCTP |
| | AAP41037.1 | PALNCYWPLN DYGFYTTTGI GYQPYRVVVL SFELLNAPAT VCGP |
| 11 | SARS-CoV-2 | MFVFLVLLPL VSSQCVNLTT RTQLPPAYTN SFTRGVYYPD KVFRSSVLHS |
| | surface | TQDLFLPFFS NVTWFHAIHV SGTNGTKRFD NPVLPENDGV YFASTEKSNI |
| | glycoprotein | IRGWIFGTTL DSKTQSLLIV NNATNVVIKV CEFQFCNDPF LGVYYHKNNK |
| | GenBank | SWMESEFRVY SSANNCTFEY VSQPFLMDLE GKQGNFKNLR EFVFKNIDGY |
| | QHD43416.1 | FKIYSKHTPI NLVRDLPQGF SALEPLVDLP IGINITRFQT LLALHRSYLT |
| | | PGDSSSGWTA GAAAYYVGYL QPRTFLLKYN ENGTITDAVD CALDPLSETK |
| | | CTLKSFTVEK GIYQTSNFRV QPTESIVRFP NITNLCPFGE VFNATRFASV |
| | | YAWNRKRISN CVADYSVLYN SASFSTFKCY GVSPTKLNDL CFTNVYADSF |
| | | VIRGDEVRQI APGQTGKIAD YNYKLPDDFT GCVIAWNSNN LDSKVGGNYN |
| | | YLYRLFRKSN LKPFERDIST EIYQAGSTPC NGVEGENCYF PLQSYGFQPT |
| | | NGVGYQPYRV VVLSFELLHA PATVCGPKKS TNLVKNKCVN FNENGLTGTG |
| | | VLTESNKKFL PFQQFGRDIA DTTDAVRDPQ TLEILDITPC SFGGVSVITP |
| | | GTNTSNQVAV LYQDVNCTEV PVAIHADQLT PTWRVYSTGS NVFQTRAGCL |
| | | IGAEHVNNSY ECDIPIGAGI CASYQTQTNS PRRARSVASQ SIIAYTMSLG |
| | | AENSVAYSNN SIAIPTNFTI SVTTEILPVS MTKTSVDCTM YICGDSTECS |
| | | NLLLQYGSFC TQLNRALTGI AVEQDKNTQE VFAQVKQIYK TPPIKDFGGF |
| | | NFSQILPDPS KPSKRSFIED LLENKVTLAD AGFIKQYGDC LGDIAARDLI |
| | | CAQKFNGLTV LPPLLTDEMI AQYTSALLAG TITSGWTFGA GAALQIPFAM |
| | | QMAYRFNGIG VTQNVLYENQ KLIANQFNSA IGKIQDSLSS TASALGKLQD |
| | | VVNQNAQALN TLVKQLSSNF GAISSVLNDI LSRLDKVEAE VQIDRLITGR |
| | | LQSLQTYVTQ QLIRAAEIRA SANLAATKMS ECVLGQSKRV DFCGKGYHLM |
| | | SFPQSAPHGV VFLHVTYVPA QEKNFTTAPA ICHDGKAHFP REGVFVSNGT |
| | | HWFVTQRNFY EPQIITTDNT FVSGNCDVVI GIVNNTVYDP LQPELDSFKE |
| | | ELDKYFKNHT SPDVDLGDIS GINASVVNIQ KEIDRLNEVA KNLNESLIDL |
| | | QELGKYEQYI KWPWYIWLGF IAGLIAIVMV TIMLCCMTSC CSCLKGCCSC |
| | | GSCCKFDEDD SEPVLKGVKL HYT |
| 12 | SARS-CoV surface | MFIFLLFLTL TSGSDLDRCT TEDDVQAPNY TQHTSSMRGV YYPDEIFRSD |
| | glycoprotein | TLYLTQDLFL PFYSNVTGFH TINHTFGNPV IPFKDGIYFA ATEKSNVVRG |
| | GenBank | WVFGSTMNNK SQSVIIINNS TNVVIRACNF ELCDNPFFAV SKPMGTQTHT |
| | AAP41037.1 | MIFDNAFNCT FEYISDAFSL DVSEKSGNFK HLREFVFKNK DGFLYVYKGY |
| | | QPIDVVRDLP SGENTLKPIF KLPLGINITN FRAILTAFSP AQDIWGTSAA |
| | | AYFVGYLKPT TFMLKYDENG TITDAVDCSQ NPLAELKCSV KSFEIDKGIY |
| | | QTSNFRVVPS GDVVRFPNIT NLCPFGEVEN ATKFPSVYAW ERKKISNCVA |
| | | DYSVLYNSTF FSTFKCYGVS ATKLNDLCFS NVYADSFVVK GDDVRQIAPG |
| | | QTGVIADYNY KLPDDEMGCV LAWNTRNIDA TSTGNYNYKY RYLRHGKLRP |
| | | FERDISNVPF SPDGKPCTPP ALNCYWPLND YGFYTTTGIG YQPYRVVVLS |
| | | FELLNAPATV CGPKLSTDLI KNQCVNFNFN GLTGTGVLTP SSKRFQPFQQ |
| | | FGRDVSDFTD SVRDPKTSEI LDISPCAFGG VSVITPGTNA SSEVAVLYQD |
| | | VNCTDVSTAI HADQLTPAWR IYSTGNNVFQ TQAGCLIGAE HVDTSYECDI |
| | | PIGAGICASY HTVSLLRSTS QKSIVAYTMS LGADSSIAYS NNTIAIPTNF |
| | | SISITTEVMP VSMAKTSVDC NMYICGDSTE CANLLLQYGS FCTQLNRALS |
| | | GIAAEQDRNT REVFAQVKQM YKTPTLKYFG GFNFSQILPD PLKPTKRSFI |
| | | EDLLFNKVTL ADAGFMKQYG ECLGDINARD LICAQKENGL TVLPPLLTDD |
| | | MIAAYTAALV SGTATAGWTF GAGAALQIPF AMQMAYRENG IGVTQNVLYE |
| | | NQKQIANQFN KAISQIQESL TTTSTALGKL QDVVNQNAQA LNTLVKQLSS |
| | | NEGAISSVLN DILSRLDKVE AEVQIDRLIT GRLQSLQTYV TQQLIRAAEI |
| | | RASANLAATK MSECVLGQSK RVDFCGKGYH LMSFPQAAPH GVVFLHVTYV |
| | | PSQERNFTTA PAICHEGKAY FPREGVEVEN GTSWFITQRN FFSPQIITTD |
| | | NTFVSGNCDV VIGIINNTVY DPLQPELDSF KEELDKYFKN HTSPDVDLGD |
| | | ISGINASVVN IQKEIDRLNE VAKNLNESLI DLQELGKYEQ YIKWPWYVWL |
| | | GFIAGLIAIV MVTILLCCMT SCCSCLKGAC SCGSCCKFDE DDSEPVLKGV |
| | | KLHYT |
| 13 | N-glycosylation at | TNLCPFGEVF NATRFASVYA WNRKNISNCV ADYSVLYNSA SFSTFKCYNV |
| | 357, 381, 386, 384, | SPTNLXDLCF TNVSADSFVI RGDEVRQIAP GQTGKIADYN YKLPDNFTGC |
| | 428 | VIAWNSNNLD SKVGGNYNYL YRLFRKSNLK PFERDISTEI YQAGSTPCNG |
| | | VEGFNCYFPL QSYGFQPTNG VGYQPYRVVV LSFELLHAPA TVCGP |
| | | wherein X is S or T |
| 14 | N-glycosylation at | TNLCPFGEVF NATRFASVYA WNRKNISNCV ADYSVLYNSA SFSTFKCYGV |
| | 357, 394, 428, 518, | SPTKLNDLCF TNVSADSFVI RGDEVRQIAP GQTGKIADYN YKLPDNFTGC |
| | 522 | VIAWNSNNLD SKVGGNYNYL YRLERKSNLK PFERDISTEI YQAGSTPCNG |
| | | VEGFNCYFPL QSYGFQPTNG VGYQPYRVVV LSFELNHXPN TXCGP |
| | | wherein X is S or T |
| 15 | N-glycosylation at | TNLCPFGEVF NATRFASVYA WNRKNISNCV ADYSVLYNSA SFSTFKCYGV |
| | 357, 394, 428, 518 | SPTKLNDLCF TNVXADSFVI RGDEVRQIAP GQTGKIADYN YKLPDNFTGC |
| | | VIAWNSNNLD SKVGGNYNYL YRLFRKSNLK PFERDISTEI YQAGSTPCNG |
| | | VEGENCYFPL QSYGFQPTNG VGYQPYRVVV LSFELNHXPA TVCGP |
| | | wherein X is S or T |
| 16 | N-glycosylation at | TNLCPFGEVF NATRFASVYA WNRKNISNCV ADYSVLYNSA SFSTFKCYGV |
| | 357, 386, 394, 428, | SPTNLXDLCF TNVXADSFVI RGDEVRQIAP GQTGKIADYN YKLPDNFTGC |
| | 518 | VIAWNSNNLD SKVGGNYNYL YRLERKSNLK PFERDISTEI YQAGSTPCNG |
| | | VEGENCYFPL QSYGFQPTNG VGYQPYRVVV LSFELNHXPA TVCGP |
| | | wherein X is S or T |
| 17 | N-glycosylation at | TNLCPFGEVF NATRFASVYA WNRKRISNCV ADYSVLYNSA SFSTFKCYGV |
| | 386, 394, 518, 522 | SPTNLXDLCF TNVXADSFVI RGDEVRQIAP GQTGKIADYN YKLPDDETGC |
| | | VIAWNSNNLD SKVGGNYNYL YRLERKSNLK PFERDISTEI YQAGSTPCNG |
| | | VEGFNCYFPL QSYGFQPTNG VGYQPYRVVV LSFELNHXPN TXCGP |
| | | wherein X is S or T |
| 18 | N-glycosylation at | TNLCPFGEVF NATRFASVYA WNRKNISNCV ADYSVLYNSA SFSTFKCYNV |
| | 357, 381, 386, 394, | SPTNLXDLCF TNVXADSFVI RGDEVRQIAP GQTGKIADYN YKLPDNFTGC |
| | 428, 518, 522 | VIAWNSNNLD SKVGGNYNYL YRLERKSNLK PFERDISTEI YQAGSTPCNG |
| | | VEGENCYFPL QSYGFQPTNG VGYQPYRVVV LSFELNHXPN TXCGP |
| | | wherein X is S or T |
| 19 | N-glycosylation at | TNLCPFGEVF NATRFASVYA WNRKNISNCV ADYSVLYNSA SFSTFKCYGV |
| | 357, 386, 394, 428, | SPTNLXDLCF TNVXADSFVI RGDEVRQIAP GQTGKIADYN YKLPDNFTGC |
| | 518, 522 | VIAWNSNNLD SKVGGNYNYL YRLFRKSNLK PFERDISTEI YQAGSTPCNG |
| | | VEGFNCYFPL QSYGFQPTNG VGYQPYRVVV LSFELNHXPN TXCGP |
| | | wherein X is S or T |
| 20 | N-glycosylation at | TNLCPFGEVF NATRFASVYA WNRKNISNCV ADYSVLYNSA SFSTFKCYNV |
| | 357, 381, 394, 518 | SPTKLNDLCF TNVXADSFVI RGDEVRQIAP GQTGKIADYN YKLPDDFTGC |
| | | VIAWNSNNLD SKVGGNYNYL YRLFRKSNLK PFERDISTEI YQAGSTPCNG |
| | | VEGFNCYFPL QSYGFQPTNG VGYQPYRVVV LSFELNHXPA TVCGP |
| | | TNVXADSFVI |
| | | wherein X is S or T |
| 21 | N-glycosylation at | TNLCPFGEVF NATRFASVYA WNRKNISNCV ADYSVLYNSA SFSTFKCYNV |
| | 357, 381, 394, 428 | SPTKLNDLCF TNVXADSFVI RGDEVRQIAP GQTGKIADYN YKLPDNFTGC |
| | | VIAWNSNNLD SKVGGNYNYL YRLFRKSNLK PFERDISTEI YQAGSTPCNG |
| | | VEGFNCYFPL QSYGFQPTNG VGYQPYRVVV LSFELLHAPA TVCGP |
| | | wherein X is S or T |
| 22 | N-glycosylation at | TNLCPFGEVF NATRFASVYA WNRKNISNCX ADYSVLYNSX SFSTEKCYNV |
| | 357, 360, 370, 381, | SPTNLXDLCF TNVXADSFVI RGDEVRQIAP GQTGKIADYN YKLPDNFTGC |
| | 386, 394, 428, 460, | VIAWNSNNLD SKVGGNYNYL YRLFRKSNLX PFERDISTEI YQAGSTPCNG |
| | 481, 503, 518, 522 | XEGENCYFPL QSYGFQPTNG NGXQPYRVVV LSFELNHXPN TXCGP |
| | | wherein X is S or T |
| 23 | SARS2_RBD_v178 | NITNLCPFGE VFNATNFSSV YAWNRKNITN CVADYSVLYN SASFSTFKCY |
| | #WT RBD plus | GVSPTKLNDL CFTNVYADSF VIRGDEVRQI APGQTGKIAD YNYKLPDNFT |
| | glycosylation sites | GCVIAWNSNN LDSKVGGNYN YLYRLERNST LKPFERDIST EIYQNGSTPC |
| | at: 346, 357, 428, | NGVNGTNCYF PLQSYGFQPT NGVGYQPYRV VVLSFELNHT PATVCGP |
| | 458, 518, 484, 475 | |
| 24 | SARS2_RBD_v183 | NITNLCPFGE VFNATNFSSV YAWNRKNITN CVADYSVLYN SASFSTFKCY |
| | #WT RBD plus | GVSPTKLNDL CFTNVYADSF VIRGDEVRQI APGQTGKIAD YNYKLPDNFT |
| | glycosylation sites | GCVIAWNSNN LDSKVGGNYN YLYRLFRKSN LKPFERDIST EIYQAGSTPC |
| | at: 346, 357, 428, 518 | NGVEGENCYF PLQSYGFQPT NGVGYQPYRV VVLSFELNHT PATVCGP |

Mutations could be added to the RBD to increase expression levels and/or increase thermal stability. The PADRE CD4 T helper epitope could be fused C-terminal to the TM domain rather than included within the extracellular tether. PADRE is an example of a T cell epitope. PADRE comprises a 13 amino acid peptide that binds with high affinity to 15 of the 16 most common human HLA-DR types, and with moderate-to-high affinity to mouse I-Ab/d and I-Eb/d MHC haplotypes and activates CD4+ T cells (Alexander et al. Development of experimental carbohydrate-conjugate vaccines composed of Streptococcus pneumoniae capsular polysaccharides and the universal helper T-lymphocyte epitope (PADRE®). Vaccine. 2004; 22(19):2362-7.) PADRE is particularly useful to overcome problems associated with polymorphism of HLA-DR molecules in the population.

Additional broadly reactive CD4 T helper epitopes could be added, either to the linker between RBD and TM, or to the C-terminus of the construct after the TM domain. Examples of such broadly reactive CD4 T helper epitopes include, without limitation: (i) The TT p2 epitope 830-QYJKANSKFJGJTE-843 (Falugi et al. 2001; SEQ ID NO:23) or the related TT epitope 826-NILMQYIKANSK-837 (Wantuch et al. 2020; SEQ ID NO:24) or those two combined into 826-NILMQYIKANSKFIGITE-843 (SEQ ID NO:25); (ii) The TT p32 epitope 1174-LKFIIKRYTPNNEIDS-1189 (Falugi et al. 2001; SEQ ID NO:26) or the related TT epitope 1169-LYNGLKFIIKR-1179 (Wantuch et al. 2020; SEQ ID NO:27) or those two combined into 1169-LYNGLKFIIKRYTPNNEIDS-1189 (SEQ ID NO:28); (iii) The CRM197 epitope 299-KTTAALSILPGIGS-312 (Wantuch et al. 2020; SEQ ID NO:29); (iv) The HBsAg_19-33 epitope FFLLTRILTIPQSLD (Celis J Immunol 1988; Greenstein et al J Virol 1992; SEQ ID NO:30); (v) The T* epitope from P. falciparum strain NF54 residues 326-345 EYLNKIQNSLSTEWSCSVT (Moreno et al. J Immunol 1993; SEQ ID NO:31).

Multimerization domains could be added in order to display clusters of the RBD on the membrane surface. While tethering to the membrane already should provide a multivalent array of RBDs for B cell interaction, it is possible that fusion to multimerization domains might enhance the local RBD density and concomitantly enhance B cell activation. Such multimerization domains could be added either to the linker between RBD and TM, or to the C-terminus of the construct after the TM domain, but preferably the multimerization domain would be added after the TM domain as this would hide the domain from B cell recognition and thus avoid generating non-RBD antibody responses. Examples of small multimerization domains (with fewer than 50 amino acids) would include trimerization motifs like the coiled-coil GCN4 (PDB ID: 1GCN) or the trimeric fibritin foldon, or tetramerization motifs like the tetrameric variant of GCN4 in PDB ID 1GCL, or the heptameric coil in PDB ID: 4PNA or the octameric coil in PDB ID: 6G67.

Larger multimerization domains with >100 amino acids that would include a larger number of CD4 T helper eptiopes could also be included. For example, a lumazine synthase domain that self-assembles into a pentamer could be fused C-terminal to the TM domain, to serve a dual purpose of providing additional T help and providing multimerization. Another example is the protein PH0250 that assembles into a 12-mer ring in PDB ID: 2EKD.

The glycan-masked RBD domains described here could be employed as immunogens in contexts other than the membrane-tethered context. For example, the glycan-masked RBD domains could be genetically fused via the N- or C-terminus to proteins that self assemble into multimeric or nanoparticle forms, and the linker between RBD and self-assembling protein could optionally include the same PADRE linker described here or it could include other CD4 T helper epitopes. Multivalent or nanoparticle presentation of antigens is well known to improve their immunogenicity.

In another example, the glycan-masked RBD domains with or without the PADRE linker could be modified to contain a cysteine residue at their N- or C-terminus and then conjugated to a phosphoserine group or chemically analogous group for the purpose of targeting the RBD to a metal hydroxide adjuvant (e.g. alum) for improved immunogenicity, following the approach of Moyer et al. Nature Medicine 26, pages 430-440 (2020).

In various aspects, the invention involves improved immunogens, including but not limited to compositions, methods of treatment, methods of making, kits and the like. In certain embodiments, the invention may comprise memRBD vaccine constructs (such as SEQ ID NO:1 or SEQ ID NO:2). In certain embodiments, the invention involves immunogens that are improved in some aspect, for example relative to memRBD vaccine constructs (such as SEQ ID NO:1 or SEQ ID NO:2) or relative to a different base immunogen. In certain embodiments the immunogen is glycan masked, such as, by for example the immunogens disclosed herein. In certain embodiments, masking is effected by mutating residues of a starting immunogen to add glycosylations sites corresponding to the glycosylation sites of the immunogens encoded in memRBD vaccine constructs (such as SEQ ID NO:1 or SEQ ID NO:2). In certain embodiments, the number of glycoslation sites added to the immunogen is one, two, three, four, five, six or more glycosylation sites. In certain embodiments, an immunogen may comprise glycosylation sites at one or more locations corresponding to glycosylation sites in memRBD vaccine constructs (such as SEQ ID NO:1 or SEQ ID NO:2). In certain embodiments, masking is effected by substituting amino acids in memRBD vaccine constructs (such as SEQ ID NO:1 or SEQ ID NO:2) disclosed herein to add glycosylation sites. In certain embodiments, masking is effected by mutating amino acids corresponding in position to those of memRBD vaccine constructs (such as SEQ ID NO:1 or SEQ ID NO:2) to increase glycosylation.

Further, in certain embodiments the antigen is engineered to display increased affinity. According to the invention, in certain embodiments, immunogens engineered for increased affinity are further engineered as to glycan masking. In a non-limiting embodiment, increased affinity immunogens, further comprise one, two, three, four, five, six or more additional glycosylation sites. In certain embodiments, the additional glycosylation sites correspond to the glycosylation sites added to memRBD vaccine constructs (such as SEQ ID NO:1 or SEQ ID NO:2). In certain increased affinity immunogens, there may be found amino acid substitutions that remove native glycosylation sites. In such cases, glycosylation sites can optionally be restored so that on or more native glycans is maintained.

The invention pertains to the identification, design, synthesis and isolation of memRBD vaccine constructs (such as SEQ ID NO:1 or SEQ ID NO:2) disclosed herein as well as nucleic acids encoding the same. The present invention also relates to homologues, derivatives and variants of the sequences of memRBD vaccine constructs (such as SEQ ID NO:1 or SEQ ID NO:2) and nucleic acids encoding the same, wherein it is preferred that the homologue, derivative or variant have at least 50%, at least 60%, at least 70%, at least 75%, at least 80%, at least 85%, at least 90%, at least 93%, at least 95%, at least 97%, at least 98% or at least 99% homology or identity with the sequence of the mutant trimers and nucleic acids encoding the same. It is noted that within this specification, homology to sequences of the mutant proteins and nucleic acids encoding the same refers to the homology of the homologue, derivative or variant to the binding site of the mutant proteins and nucleic acids encoding the same.

The invention still further relates to nucleic acid sequences expressing the mutant trimers disclosed herein, or homologues, variants or derivatives thereof. One of skill in the art will know, recognize and understand techniques used to create such. Additionally, one of skill in the art will be able to incorporate such a nucleic acid sequence into an appropriate vector, allowing for production of the amino acid sequence of mutant proteins and nucleic acids encoding the same or a homologue, variant or derivative thereof.

In another aspect, the invention provides a diagnostic serological probe. RBDs which may comprise selectively masked epitopes are advantageous for detecting epitope-specific antibodies in serum and other polyclonal mixtures. For example, masking non-nAB binding sites allows for detection of nAbs, and masking of nAb binding sites allows for detection of non-nAbs. Such reagents can comprise masked nAb and/or non-nAb epitopes in any desired combination.

In another aspect, the invention provides a diagnostic serological probe tailored to reduce receptor binding. As demonstrated herein, adding a glycosylation site at N503 of SARS-CoV-2 reduced ACE binding to undetectable levels while binding to other nAbs and non-nAbs was diminished by at most, less that a factor of 10.

Where used herein and unless specifically indicated otherwise, the following terms are intended to have the following meanings in addition to any broader (or narrower) meanings the terms might enjoy in the art:

The term “isolated” or “non-naturally occurring” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art may readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

In an advantageous embodiment, a tag may be utilized for purification or biotinylation. The tag for purification may be a his tag. In another embodiment, the tag for biotinylation may be an avi-tag. Other tags are contemplated for purification, however, purification may be accomplished without a tag. In another embodiment, antibody (such as, not limited to, a broadly neutralizing antibody) affinity columns are contemplated. In another embodiment, lectin columns are contemplated.

The proteins and compounds of the invention need not be isolated in the sense defined above, however.

The term “pharmaceutical composition” is used herein to define a solid or liquid composition in a form, concentration and level of purity suitable for administration to a patient (e.g. a human patient) upon which administration it may elicit the desired physiological changes. The terms “immunogenic composition” and “immunological composition” and “immunogenic or immunological composition” cover any composition that elicits an immune response against the targeted pathogen, SARS-CoV-2. Terms such as “vaccinal composition” and “vaccine” and “vaccine composition” cover any composition that induces a protective immune response against the targeted pathogen or which efficaciously protects against the pathogen or virus; for instance, after administration or injection, elicits a protective immune response against the targeted pathogen or virus or provides efficacious protection against the pathogen or virus. Accordingly, an immunogenic or immunological composition induces an immune response, which may, but need not, be a protective immune response. An immunogenic or immunological composition may be used in the treatment of individuals infected with the pathogen or virus, e.g., to stimulate an immune response against the pathogen, such as by stimulating antibodies against the pathogen or virus. Thus, an immunogenic or immunological composition may be a pharmaceutical composition. Furthermore, when the text speaks of “immunogen, antigen or epitope”, an immunogen may be an antigen or an epitope of an antigen. A diagnostic composition is a composition containing a compound or antibody, e.g., a labeled compound or antibody, that is used for detecting the presence in a sample, such as a biological sample, e.g., blood, semen, vaginal fluid, etc., of an antibody that binds to the compound or an immunogen, antigen or epitope that binds to the antibody; for instance, an anti-SARS-CoV-2 antibody or a SARS-CoV-2 immunogen, antigen or epitope.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged amino acids or polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
(a) Fab, the fragment which contains a monovalent antigen-binding fragment of an antibody molecule may be produced by digestion of whole antibody with the enzyme papain to yield an intact light chain and a portion of one heavy chain;
(b) Fab′, the fragment of an antibody molecule may be obtained by treating whole antibody with pepsin, followed by reduction, to yield an intact light chain and a portion of the heavy chain; two Fab′ fragments are obtained per antibody molecule;
(c) F(ab′)₂, the fragment of the antibody that may be obtained by treating whole antibody with the enzyme pepsin without subsequent reduction; F(ab′)₂ is a dimer of two Fab′ fragments held together by two disulfide bonds;
(d) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference). Fabs, Fv and scFV may also be made recombinantly, i.e. expressed as Fab, Fv or scFV rather than cleaving an intact IgG.

A “neutralizing antibody” may inhibit the entry of SARS-CoV-2 virus for example SF162 and/or JR-CSF with a neutralization index >1.5 or >2.0. Broad and potent neutralizing antibodies may neutralize greater than about 50% of SARS-CoV-2 viruses (from diverse clades and different strains within a clade) in a neutralization assay. The inhibitory concentration of the monoclonal antibody may be less than about 25 mg/ml to neutralize about 50% of the input virus in the neutralization assay.

An “isolated antibody” or “non-naturally occurring antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “monoclonal antibody” as used herein refers to an antibody obtained from a population of substantially homogeneous antibodies, i.e., the individual antibodies which may comprise the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

An “antibody fragment” may comprise a portion of an intact antibody, preferably the antigen binding or variable region of the intact antibody. Examples of antibody fragments include Fab, Fab′, F(ab′)₂, scFV and Fv fragments; diabodies; linear antibodies (see U.S. Pat. No. 5,641,870; Zapata et al. 1995 Protein Eng. 8(10): 1057-1062); single-chain antibody molecules; and multispecific antibodies formed from antibody fragments.

It should be understood that the proteins, including the antibodies of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated or non-naturally occurring replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double-stranded (duplex). Duplex nucleic acids may be homoduplex or heteroduplex.

As used herein the term “transgene” may used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the sequences of the invention, such as the mutant trimers, may be altered in these ways.

As regards codon optimization, the nucleic acid molecules of the invention have a nucleotide sequence that encodes the antigens of the invention and may be designed to employ codons that are used in the genes of the subject in which the antigen is to be produced. Many viruses use a large number of rare codons and, by altering these codons to correspond to codons commonly used in the desired subject, enhanced expression of the antigens may be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by SARS-CoV-2. Such codon usage provides for efficient expression of the transgenic SARS-CoV-2 proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention may readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms may be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and antibodies of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the proteins of the present invention may be used in accordance with the present invention. In certain embodiments, the proteins of the present invention may be used in vitro (such as using cell-free expression systems) and/or in cultured cells grown in vitro in order to produce the encoded SARS-CoV-2 proteins, which may then be used for various applications such as in the production of proteinaceous vaccines. For such applications, any vector that allows expression of the proteins in vitro and/or in cultured cells may be used.

For applications where it is desired that the proteins be expressed in vivo, for example when the transgenes of the invention are used in DNA or DNA-containing vaccines, any vector that allows for the expression of the proteins of the present invention and is safe for use in vivo may be used. In preferred embodiments the vectors used are safe for use in humans, mammals and/or laboratory animals.

For the proteins of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ. Many suitable promoters and enhancers are known in the art, and any such suitable promoter or enhancer may be used for expression of the transgenes of the invention. For example, suitable promoters and/or enhancers may be selected from the Eukaryotic Promoter Database (EPDB).

The vectors used in accordance with the present invention should typically be chosen such that they contain a suitable gene regulatory region, such as a promoter or enhancer, such that the antibodies of the invention may be expressed.

Any suitable vector may be used depending on the application. For example, plasmids, viral vectors, bacterial vectors, protozoal vectors, insect vectors, baculovirus expression vectors, yeast vectors, mammalian cell vectors, and the like, may be used. Eucaryotic expression vectors are advantageous. Suitable vectors may be selected by the skilled artisan taking into consideration the characteristics of the vector and the requirements for expressing the proteins under the identified circumstances.

Volz describes a recombinant modified vaccinia virus Ankara (MVA) vaccine expressing full-length MERS-CoV spike (S) glycoprotein and immunizing BALB/c mice with either intramuscular or subcutaneous regimens. (Volz et al., Protective Efficacy of Recombinant Modified Vaccinia Virus Ankara Delivering Middle East Respiratory Syndrome Coronavirus Spike Glycoprotein. Journal of Virology July 2015, 89 (16) 8651-8656; DOI: 10.1128/JVI.00614-15). Such a vaccine is useful to express immunogens disclosed herein.

Malczyk generated MVs expressing the spike glycoprotein of MERS-CoV in its full-length (MERS-S) or a truncated, soluble variant of MERS-S. (Malczyk et al., A Highly Immunogenic and Protective Middle East Respiratory Syndrome Coronavirus Vaccine Based on a Recombinant Measles Virus Vaccine Platform. Journal of Virology October 2015, 89 (22) 11654-11667; DOI: 10.1128/JVI.01815-15). The engineered glycoporteins of the invention can be similarly expressed.

Wang generated MERS-CoV VLPs using the baculovirus expression system. Innoculation of Rhesus macaques with MERS-CoV VLPs and Alum adjuvant induced virus-neutralizing antibodies against the RBD. (Wang et al., MERS-CoV virus-like particles produced in insect cells induce specific humoural and cellular imminity in rhesus macaques. Oncotarget. 2017 Feb. 21; 8(8): 12686-12694. Published online 2016 Mar. 30. doi: 10.18632/oncotarget.8475) The immunogens provided herein can be similarly experssed from a baculovirus expression system and used for immunozation.

McPherson describes methods for expression, purification, release testing, adjuvant formulation, and animal testing of SARS recombinant spike protein antigen. (McPherson et al., Development of a SARS Coronavirus Vaccine from Recombinant Spike Protein Plus Delta Inulin Adjuvant. All of the vaccine compositions of the present invention can be produced, formulated, and tested accordingly. In: Vaccine Design Methods and Protocols: Volume 1: Vaccines for Human Diseases, Editors: Sunil Thomas DOI: 10.1007/978-1-4939-3387-7_14). All of the vaccine compositions of the present invention can be produced, formulated, and tested accordingly.

Du reports a recombinant adeno-associated virus (rAAV)-based RBD (RBD-rAAV) vaccine could induce highly potent neutralizing Ab responses in immunized animals. (Du et al., Intranasal Vaccination of Recombinant Adeno-Associated Virus Encoding Receptor-Binding Domain of Severe Acute Respiratory Syndrome Coronavirus (SARS-CoV) Spike Protein Induces Strong Mucosal Immune Responses and Provides Long-Term Protection against SARS-CoV Infection. J Immunol Jan. 15, 2008, 180 (2) 948-956; DOI: 10.4049/jimmunol.180.2.948).

Liu describes constructs and methods for a chimeric virus based on the vesicular stomatitis virus (VSV) in which the G gene was replaced by MERS-CoV S gene and reports the S protein efficiently incorporated into the viral envelope and mediated cell entry through binding its receptor, human DPP4. (Liu et al. A recombinant VSV-vectored MERS-CoV vaccine induces neutralizing antibody and T cell responses in rhesus monkeys after single dose immunization. Antiviral Research. 150:30-38, February 2018, DOI: 10.1016/j.antiviral.2017.12.007).

The glycoprotein immunogens disclosed herein can likewise be expressed using a recombinant adeno-associated virus system.

Darnel reports inactivation methods which allow SARS-containing materials to be rendered non-infectious. (Darnell et al., Inactivation of the coronavirus that induces severe acute respiratory syndrome, SARS-CoV. J Virol Methods. 2004 October; 121(1): 85-91.) The same methods are used with the instant glycoproteins and viral expression systems.

Hervas-Stubbs has reported the general applicability of Baculoviruses (BVs) for immunization, including that BVs have strong adjuvant properties in mice, promoting potent humoral and CD8+ T cell adaptive responses against coadministered Ag. (Hervas-Stubbs et al., Insect Baculoviruses Strongly Potentiate Adaptive Immune Responses by Inducing Type I IFN. J Immunol Feb. 15, 2007, 178(4) 2361-2369; DOI: 10.4049/jimmunol.178.4.2361).

Moss (WO2006071250A2) reported an attenuated poxvirus carrying a spike (S) polypeptide induces formation of neutralizing antibodies and protectively immunizes animals against a subsequent infection with SARS-CoV. Antiserum collected from animals immunized with the attenuated poxvirus reduced SARS viral replication in infected animals. As also described herein, a secreted, glycosylated S polypeptide including amino acids 14 to 762 of the SARS coronavirus (SARS-CoV) S protein provided complete protection of the upper and lower respiratory tract against SARS infection. Poxviruses replicate entirely in the cytoplasm. They have been used as vaccines since the early 1980's (see, e.g., Panicali, D. et al. Construction of live vaccines by using genetically engineered pox viruses: biological activity of recombinant vaccinia virus expressing influenza virus hemagglutinin, Proc. Natl. Acad. Sci. USA 80:5364-5368, 1983).

Sutter (WO2016116398A1) reports development of vaccines and compositions to protect from MERS-CoV infection. Viruses of Modified Vaccinia virus Ankara (MVA) stably containing gene sequences encoding the full-length MERS-CoV proteins S and N were constructed. The recombinant MVA viruses amplified to high titers in chicken embryo fibroblasts and intramuscular vaccination of BALB/c mice with MVA-MERS-SN confirmed the particular immunogenicity of the recombinant N protein. Vaccination raised high levels of serum antibodies that reacted with the authentic N protein of MERS-CoV.

Liu reports a recombinant MERS-CoV vaccine elicits high-level and lasting neutralizing antibodies in camels. The authors used recombinant nonvirulent Newcastle disease virus (NDV) LaSota strain expressing MERS-CoV S protein. (Liu et al., Newcastle disease virus-based MERS-CoV candidate vaccine elicits high-level and lasting neutralizing antibodies in Bactrian camels. Journal of Integrative Agriculture, October 2017, 16(10,):2264-2273). All of the technologies and methods reported above for coronavirus are generally applicable to the engineered RBD-containing glycoproteins disclosed herein.

Wang reviews the development of mRNA-based SARS-CoV-2 vaccines, including non-replicating mRNA vaccines and self-amplifying or replicon RNA vaccines and different delivery methods, such as ex vivo loading of dendritic cell and direct in vivo injection into various anatomical sites. (Wang et al., An Evidence Based Perspective on mRNA-SARS-CoV-2 Vaccine Development. Med Sci Monit. 2020; 26: e924700-1-e924700-8. doi: 10.12659/MSM.924700).

Inovio has announced positive results from the first-in-human trial of its DNA vaccine against MERS. (Modjarrad et al., Safety and immunogenicity of an anti-Middle East respiratory syndrome coronavirus DNA vaccine: A phase 1, open-label, single-arm, dose-escalation trial, The Lancet, Sep. 1, 2019, 19(9)1013-1022; see also Clinical trial NCT0371718: Evaluate the Safety, Tolerability and Immunogenicity Study of GLS-5300 in Healthy Volunteers). The present glycoprotein compositions, vaccines and methods can be tested and administered by the same procedures.

Generally, mammalian expression systems producing mammalian N-linked glycans are preferred, the goal being to promote neutralizing immune responses involving selected SARS-CoV-2 epitopes while minimize immunogenicity of epitopes that would elicit non-neutralizing antibodies. In certain instances, human expression systems such as HEK293 may be preferred, as animal cells such as CHO, Sp2/0 and NS0 mouse myeloma cells can produce glycoproteins with non-human glycans that may potentially illicit immunogenic responses.

Goh describes the types of host cells used for production of therapeutics, their glycosylation potential and the resultant impact on glycoprotein properties. Goh describes the various complex-type N-linked glycans and commonly used mammalian production cells, including Chinese hamster ovary (CHO) cells, baby hamster kidney (BHK) cells, NS0 myeloma and Sp2/0 hybridoma mouse cell lines, human embryonic kidney cells 293 (HEK293) and HT-1080 human cells. (Goh et al., Impact of host cell line choice on glycan profile. Critical Reviews in Biotechnology, 2008, 38:6, 851-867, DOI: 10.1080/07388551.2017.1416577). Lalonde reviews mammalian cell lines, including considerations of cell types, cell engineering, gene expression, cell growth and proliferation, protein folding and secretion. (Lalonde et al., Therapeutic glycoprotein production in mammalian cells. Journal of Biotechnology, 2017, 251:128; 10.1016/j.jbiotec.2017.04.028). The glycoprotein compostions are expressed similarly, taking in to account, for example, variations in types of glycosylation, protein expression and the like.

Croset examined differences in glycosylation in mammalian cells of 12 proteins in the two commonly used cell lines HEK and CHO. The cells were transiently transfected, and the expressed proteins were purifiedand analyzed on SDS-PAGE, isoelectric focusing (IEF), mass spectrometry and released glycans on capillary gel electrophoresis (CGE-LIF). For all proteins significant differences in the glycosylation were detected. The proteins migrated differently on SDS-PAGE, had different isoform patterns on IEF, showed different mass peak distributions on mass spectrometry and showed differences in the glycostructures detected in CGE. (Croset et al., Differences in the Glycosylation of Recombinant Proteins Expressed in HEK and CHO Cells. J Biotechnol. 2012 Oct. 31; 161(3):336-48. doi: 10.1016/j.jbiotec.2012.06.038. Epub 2012 Jul. 16.)

Clausen describes glycan structures and their metabolism, including state of glycosylation engineering in different cell types using precise gene-editing technologiesl (Clausen et al, Essentials of Glycobiology. 3rd edition. Varki A, Cummings R D, Esko J D, et al., editors. Cold Spring Harbor (NY): Cold Spring Harbor Laboratory Press; 2015-2017). Butler points out that although the glycosylation profile of these products is ‘human-like’ there is still the possibility of immunogenic epitopes such as α-Gal and Neu5Gc, and describes Human cell lines designed for high productivity of recombinant proteins and ensuring authentic glycosylation patterns. (Butler et al., The choice of mammalian cell host and possibilities for glycosylation engineering. Current Opinion in Biotechnology 2014, 30:107-112. DOI: 10.1016/j.copbio.2014.06.010).

Hunter reviews strategies for producing recombinant proteins in mammalian cell lines to introduce proper protein folding and post-translational modifications and how to overcome various obstacles that may be encountered. (Hunter et al., Optimization of Protein Expression in Mammalian Cells. Current Protocols, February 2019, Volume 95, Issue 1, p. e77. DOI:10.1002/cpps.77). Gupta reviews approaches that minimize the glycan heterogeneity for the production of the desired protein with improved glycoforms, including mammalian, insect, and yeast and glycoengineering to produce human-like glycan composition of a recombinant product. Gupta et al., Glycosylation control technologies for recombinant therapeutic proteins. Appl Microbiol Biotechnol 2018, 102, 10457-10468). The procedures and improvements described by Clausen and Hunter are used in expressing the glycoproteins of the invention

When the aim is to express the proteins of the invention in vivo in a subject, for example in order to generate an immune response against a SARS-CoV-2 antigen and/or protective immunity against SARS-CoV-2, expression vectors that are suitable for expression on that subject, and that are safe for use in vivo, should be chosen. For example, in some embodiments it may be desired to express the proteins of the invention in a laboratory animal, such as for pre-clinical testing of the SARS-CoV-2 immunogenic compositions and vaccines of the invention. In other embodiments, it will be desirable to express the proteins of the invention in human subjects, such as in clinical trials and for actual clinical use of the immunogenic compositions and vaccine of the invention. Any vectors that are suitable for such uses may be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Vesicular stomatitis viruses (VSV) are also contemplated, especially if the VSV G protein is substituted with another protein, such as the memRBD of the present invention. Such viruses, when used as expression vectors are innately non-pathogenic in the selected subjects such as humans or have been modified to render them non-pathogenic in the selected subjects. For example, replication-defective adenoviruses and alphaviruses are well known and may be used as gene delivery vectors.

The nucleotide sequences and vectors of the invention may be delivered to cells, for example if the aim is to express the SARS-CoV-2 antigens in cells in order to produce and isolate the expressed proteins, such as from cells grown in culture. For expressing the antibodies in cells any suitable transfection, transformation, or gene delivery methods may be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies may be carried out in any suitable type of host cells, such as bacterial cells, yeast, insect cells, and mammalian cells. The antibodies of the invention may also be expressed using including in vitro transcription/translation systems. All of such methods are well known by those skilled in the art, and one of skill in the art would readily be able to select a suitable method depending on the nature of the nucleotide sequences, vectors, and cell types used.

A synthetic mutant trimer may be chemically synthesized in whole or part using techniques that are well-known in the art (see, e.g., Kochendoerfer, G. G., 2001). Additionally, homologs and derivatives of the polypeptide may be also be synthesized.

Alternatively, methods which are well known to those skilled in the art may be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989.

Pre-clinical vaccine testing models, in particular the nucleic acid delivery systems, may be adapted to express the memRBD of the present invention.

J. Yu et al., Science 10.1126/science. abc6284 (2020) reports a naked DNA vaccine immunization in NHPs with various SARS-CoV-2 constructs, including a trimerized RBD, and tests protection against a virus challenge.

Corbett et al. (bioRxiv preprint https://doi.org/10.1101/2020.06.11.145920) report development of a Moderna mRNA vaccine called mRNA-1273 that encodes a stabilized SARS-CoV-2 spike which shows elicitation of nAbs and CD8 responses in mice.

McKay et al. describe a self-amplifying RNA nanoparticle vaccine to immunize mice with saRNA encoding the SARS-CoV-2 spike protein encapsulated in LNP with doses ranging from 0.01 to 10 μg. (McKay et al., Self-amplifying RNA SARS-CoV-2 lipid nanoparticle vaccine induces equivalent preclinical antibody titers and viral neutralization to recovered COVID-19 patients. bioRxiv preprint doi: 10.1101/2020.04.22.055608 posted Apr. 25, 2020.) The methods disclosed can be used to express RBDs and proteins of the invention.

Erasmus describes a highly immunogenic vaccine candidate comprised of an RNA replicon (LION) designed to enhance vaccine stability, delivery, and immunogenicity delivery and immunogenicity for intramuscular injection to elicit antibody and T cell responses to SARS-CoV-2. (Erasmus et al., Single-dose replicating RNA vaccine induces neutralizing antibodies against SARS-CoV-2 in nonhuman primates. bioRxiv preprint doi: 10.1101/2020.05.28.121640 posted May 28, 2020). The methods disclosed can be used with the instant invention.

Smith et al (NATURE COMMUNICATIONS1 (2020) 11:2601 |https://doi.org/10.1038/s41467-020-16505-0|www.nature.com/naturecommunications) report mouse immunization results with the Inovio DNA vaccine encoding a SARS-CoV-2 spike protein.

Quinlan et al (bioRxiv preprint https://doi.org/10.1101/2020.04.10.036418doi) test a subunit vaccine composed of RBD-Fc conjugated to KLH carrier protein tests in rodents. Quinlan et al. show that the prototype elicits neutralizing antibodies and those antibodies do not mediate antibody-dependent enhancement (ADE) under conditions in which Zika virus ADE had previously been observed.

Ravichandran et al (bioRxiv preprint https://doi.org/10.1101/2020.05.12.091918) test three different subunit vaccines, including spike ectodomain (S1+S2), S1, and RBD, in rabbits and show that all three subunit vaccines elicit neutralizing Abs.

van Doremalen et al (bioRxiv preprint. https://doi.org/10.1101/2020.05.13.093195) report the Oxford vaccine that is being used by AstraZeneca (ChAdOx1 Chimpanzee Adenovirus vector (ChAd)) show that this vaccine provides some protection in NHPs.

Other vaccine modalities that could express the glycan-masked RBD of the present invention, but without the TM tether, may include the below.

Moyer et al (NATURE MEDICINE|VOL 26|March 2020|430-440) show how to add a phosphoserine motif to a subunit vaccine which then mediates binding to Alum, which this enhances in vivo trafficking to LNs and enhances immunogenicity.

Several examples of vaccine platforms (self-assembling RBD-NPs) that could present a glycan-masked RBD for SARS-CoV-2 are presented below.

Jardine et al (SCIENCE VOL 340 10 May 2013) report a design of a self-assembling nanoparticle presenting an engineered outer domain from HIV (eOD-GT6 60mer).

Sok et al. (SCIENCE 30 Sep. 2016 VOL 353 ISSUE 6307) show that the next generation version of this nanoparticle (eOD-GT8 60mer) induces responses from rare precursors in human-Ig-transgenic mice.

Kanekiyo et al. (NATURE IMMUNOLOGY|VOL 20|March 2019|362-372) test purified protein subunit vaccines that are self-assembling nanoparticles (NPs) presenting RBDs from influenza hemagglutinin that elicit neutralizing responses.

Xu et al. describe a DNA vaccine comprising self-assembling nanoparticles comprising lumazine synthase for vaccination with an HIV immunogen and induction of strong humoral responses. (Xu et al., In Vivo Assembly of Nanoparticles Achieved through Synergy of Structure-Based Protein Engineering and Synthetic DNA Generates Enhanced Adaptive Immunity. Adv. Sci. 2020, DOI: 10.1002/advs.201902802. The methods used by Xu are applicable to vaccination with glycan-masked RBDs if the invention.

Melo et al. describe an alphavirus RNA replicon for vaccination of subjects with germline-targeting HIV immunogens. (Melo et al., Immunogenicity of RRNA Replicons Encoding HIV Env Immunogens Designed for Self-Assembly into Nanoparticles. Molecular Therapy, Vol. 27 No 12, pp. 1-11, December 2019.) The methods disclosed can be used to express RBDs and coronavirus surface glycoproteins of the invention.

The invention also encompasses eliciting an immune response which may comprise systemically administering to an animal in need thereof an effective amount of any one of the non-naturally occurring protein(s) of the invention or a nucleic acid(s) encoding the same. The animal may be a mammal, advantageously a human.

In one embodiment, the nucleic acids of the present invention may be delivered as a therapeutic mRNA.

Provided herein are isolated nucleic acids (e.g., modified mRNAs encoding a peptide described herein) which may comprise a translatable region and at least two different nucleoside modifications, wherein the nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. In certain embodiments, the nucleic acid may comprise RNA, DNA, TNA, GNA, or a hybrid thereof. In certain embodiments, the nucleic acid may comprise messenger RNA (mRNA). In certain embodiments, the mRNA does not substantially induce an innate immune response of the cell into which the mRNA is introduced. In certain embodiments, the mRNA may comprise at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In certain embodiments, the mRNA may comprise at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, the mRNA may comprise at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In yet other embodiments, the mRNA may comprise at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleic acids provided herein comprise a 5′ untranslated region (UTR) and/or a 3′UTR, wherein each of the two different nucleoside modifications are independently present in the 5′UTR and/or 3′UTR. In some embodiments, nucleic acids are provided herein, wherein at least one of the two different nucleoside modifications are present in the translatable region. In some embodiments, nucleic acids provided herein are capable of binding to at least one polypeptide that prevents or reduces an innate immune response of a cell into which the nucleic acid is introduced.

Further provided herein are isolated nucleic acids (e.g., modified mRNAs described herein) which may comprise (i) a translatable region encoding a peptide described herein, (ii) at least one nucleoside modification, and (iii) at least one intronic nucleotide sequence capable of being excised from the nucleic acid.

Further provided herein are isolated nucleic acids (e.g., modified mRNAs described herein) which may comprise (i) a translatable region encoding a peptide described herein, (ii) at least two different nucleoside modifications, and (iii) a degradation domain.

Further provided herein are non-enzymatically synthesized nucleic acids (e.g., modified mRNAs described herein) which may comprise at least one nucleoside modification, and which may comprise a translatable region encoding a peptide described herein. In certain embodiments, the non-enzymatically synthesized mRNA may comprise at least two different nucleoside modifications.

Further provided herein are isolated nucleic acids (e.g., modified mRNAs described herein) which may comprise a noncoding region and at least one nucleoside modification that reduces an innate immune response of a cell into which the nucleic acid is introduced, wherein the nucleic acid sequesters one or more translational machinery components. In certain embodiments, the isolated nucleic acids which may comprise a noncoding region and at least one nucleoside modification described herein are provided in an amount effective to reduce protein expression in the cell. In certain embodiments, the translational machinery component is a ribosomal protein or a transfer RNA (tRNA). In certain embodiments, the nucleic acid may comprise a small nucleolar RNA (sno-RNA), microRNA (miRNA), small interfering RNA (siRNA) or Piwi-interacting RNA (piRNA).

Further provided herein are isolated nucleic acids (e.g., modified mRNAs described herein) which may comprise (i) a first translatable region, (ii) at least one nucleoside modification, and (iii) an internal ribosome entry site (IRES). In certain embodiments, the IRES is obtained from a picornavirus, a pest virus, a polio virus, an encephalomyocarditis virus, a foot-and-mouth disease virus, a hepatitis C virus, a classical swine fever virus, a murine leukemia virus, a simian immune deficiency virus or a cricket paralysis virus. In certain embodiments, the isolated nucleic acid further may comprise a second translatable region. In certain embodiments, the isolated nucleic acid further may comprise a Kozak sequence. In some embodiments, the first translatable region encodes a peptide described herein. In some embodiments, the second translatable region encodes peptide described herein. In some embodiments, the first and the second translatable regions encode peptides described herein.

Provided herein are pharmaceutical compositions which may comprise: (i) an effective amount of a synthetic messenger ribonucleic acid (mRNA) encoding peptide described herein; and (ii) a pharmaceutically acceptable carrier, wherein i) the mRNA may comprise pseudouridine, 5′methyl-cytidine, or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to a mammalian subject in need thereof. In some embodiments,

Further provided herein are pharmaceutical compositions which may comprise and/or consisting essentially of: (i) an effective amount of a synthetic messenger ribonucleic acid (mRNA) encoding peptide described herein; (ii) a cell penetration agent; and (iii) a pharmaceutically acceptable carrier, wherein i) the mRNA may comprise pseudouridine, 5′methyl-cytidine or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to an animal (e.g., mammalian) subject in need thereof.

This invention provides nucleic acids, including RNAs such as mRNAs that contain one or more modified nucleosides (termed “modified nucleic acids”), which have useful properties including the lack of a substantial induction of the innate immune response of a cell into which the mRNA is introduced. Because these modified nucleic acids enhance the efficiency of protein production, intracellular retention of nucleic acids, and viability of contacted cells, as well as possess reduced immunogenicity, these nucleic acids having these properties are termed “enhanced nucleic acids” herein.

The term “nucleic acid,” in its broadest sense, includes any compound and/or substance that is or can be incorporated into an oligonucleotide chain. Exemplary nucleic acids for use in accordance with the present invention include, but are not limited to, one or more of DNA, RNA, hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein.

Provided are modified nucleic acids containing a translatable region encoding a peptide described herein, and one, two, or more than two different nucleoside modifications. In some embodiments, the modified nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or a hybrid thereof. In preferred embodiments, the modified nucleic acid includes messenger RNAs (mRNAs). As described herein, the nucleic acids of the invention do not substantially induce an innate immune response of a cell into which the mRNA is introduced.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In certain embodiments it is desirable to intracellularly degrade a modified nucleic acid introduced into the cell, for example if precise timing of protein production is desired. Thus, the invention provides a modified nucleic acid containing a degradation domain, which is capable of being acted on in a directed manner within a cell.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Other components of nucleic acid are optional, and are beneficial in some embodiments. For example, a 5′ untranslated region (UTR) and/or a 3′UTR are provided, wherein either or both may independently contain one or more different nucleoside modifications. In such embodiments, nucleoside modifications may also be present in the translatable region. Also provided are nucleic acids containing a Kozak sequence.

Further, nucleic acids encoding a peptide described herein, and containing an internal ribosome entry site (IRES) are provided herein. An IRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

The therapeutic mRNAs as described, for example, in U.S. Pat. Nos. 9,464,124; 9,447,164; 9,428,535; 9,334,328; 9,303,079; 9,301,993; 9,295,689; 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,822,663; 8,754,062; 8,710,200; 8,680,069 and 8,664,194 may be utilized for the present invention.

Methods for the chemical conjugation of polypeptides, carbohydrates, and/or lipids are well known in the art (see, for example, Hermanson. Bioconjugate Techniques (Academic Press; 1992); Aslam and Dent, eds. Bioconjugation: Protein coupling Techniques for the Biomedical Sciences (MacMillan: 1998); and Wong Chemistry of Protein Conjugation and Cross-linking (CRC Press: 1991)). For instance, primary amino groups may be incorporated by reaction with ethylenediamine in the presence of sodium cyanoborohydride and sulfhydryls may be introduced by reaction of cysteamin dihydrochloride followed by reduction with a standard disulfide reducing agent. Heterobifunctional crosslinkers, such as, for example, sulfosuccinimidyl (4-iodoacetyl) aminobenzoate, which link the epsilon amino group on the D-lysine residues of copolymers of D-lysine and D-glutamate to a sulfhydryl side chain from an amino terminal cysteine residue on the peptide to be coupled, may be used as well. Chemical conjugation also includes anything covalently bonded directly via side chain bonds or via a linker or spacer group.

The nanoparticle formulations may be a carbohydrate nanoparticle which may comprise a carbohydrate carrier and a modified nucleic acid molecule (e.g., mmRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

The average diameter of the nanoparticle employed in the compositions of the invention can be at least one member selected from the group consisting of about 20 nanometers, about 25 nanometers, about 30 nanometers, about 40 nanometers, about 50 nanometers, about 75 nanometers, about 100 nanometers, about 125 nanometers, about 150 nanometers, about 175 nanometers and about 200 nanometers. In another embodiment, the average diameter of the particle is at least one member selected from the group consisting of between about 10 to about 200 nanometers, between about 0.5 to about 5 microns and between about 5 to about 10 microns. In another embodiment, the average diameter of the microparticle is selected from the group consisting of about 0.1 μm, about 0.2 μm, about 0.4 μm, about 0.5 μm, about 1 μm and about 2 μm.

Nanoparticles for use in the compositions of the invention can be made from lipids or other fatty acids (see, for example, U.S. Pat. Nos. 5,709,879; 6,342,226; 6,090,406; Lian, et al., J. of Pharma. Sci. 90:667-680 (2001) and van Slooten, et al., Pharm Res. 17:42-48 (2000)) and non-lipid compositions (see, for example, Kreuter, J. Anat. 189:503-505 (1996), the teachings of all of which are hereby incorporated by reference in their entirety). The compositions can be bilayer or multilamellar liposomes and phospholipid based. Polymerized nanoparticles, as described, for example, in U.S. Pat. No. 7,285,289, the teachings of which are incorporated by reference in their entirety.

Metallic oxide nanoparticles for use in the compositions of the invention can be chemically substituted with at least one reactive moiety capable of forming a thioether bond employing conventionally techniques as described herein and in U.S. Pat. No. 6,086,881, the teachings of which are hereby incorporated by reference in their entirety. The antigen described herein can be coupled in a single step onto the metallic oxide particles by the formation of at least one thioether bond or it may be synthesized or assembled stepwise onto the metallic oxide particles after the initial thioether bond formation. The chemical derivatization reagents for the metallic oxide particles can include organosilane reagents that provide thioalkane functionality or other groups that may readily be converted into thiols or thiol-reactive moieties. Organosilane reagents which may be utilized for this purpose may be, but are not limited to, 3-mercaptopropyltrimethoxysilane, 3-aminopropyltriethoxysilane, 3-iodopropyltrimethoxysilane, 2-chloroethyltrichlorosilane, 3-glycidoxypropyltrimethoxysilane, vinyltrichlorosilane and 3-acryloxypropyltrimethoxysilane. Moieties that include one or more disulfide components may also be joined to the metallic oxide particle surface and thereby provide the corresponding reactive moiety able to enter into and form a thioether bond and juncture. Exemplary nanoparticles for use in the compositions of the invention include at least one member selected from the group consisting of poly (D,L-lactide-co-glycolide, also referred to as “poly(lactic-co-glycolic acid) and bisacyloxypropylcysteine.

Nanoparticles for use in the compositions of the invention can be made of inorganic material. Nanoparticles for use in the compositions of the invention can be made of a polymer material, such as at least one member selected from the group consisting of polystyrene, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, a carbohydrate, carboxymethyl cellulose, hydroxyethyl cellulose, agar, gel, proteinaceous polymer, polypeptide, eukaryotic and prokaryotic cells, viruses, lipid, metal, resin, latex, rubber, silicone (e.g., polydimethyldiphenyl siloxane), glass, ceramic, charcoal, kaolinite and bentonite.

In some embodiments, a fusion polypeptide described herein further may comprise a self-assembling domain capable of forming a nanoparticle. In some embodiments, the self-assembling domain may comprise a type II 3-Dehydroquinase, ferritin or lumazine synthase. In some embodiments, the self-assembling domain may comprise a type II 3-Dehydroquinase polypeptide which may comprise one or more engineered glycosylation site. In some embodiments, the self-assembling domain may comprise a Thermus thermophilus type type II 3-Dehydroquinase, optionally which may comprise one or more engineered glycosylation site.

In some embodiments, the self-assembling domain may comprise a type II 3-Dehydroquinase, ferritin or lumazine synthase. In some embodiments, the self-assembling domain may comprise a Thermus thermophilus, Mycobacterium tuberculosis, Streptomyces coelicolor, Acinetobacter baumannii, Yersinia pestis, Bacillus subtilis, Proprionibacterium acnes, Acidithiobacillus caldus, Zymomonas mobilus, Helicobacter pylori, Pseudomonas aeruginosa, Candida albicans, or Psychromonas ingrahamii type II 3-Dehydroquinase polypeptide.

In some embodiments, the self-assembling domain may comprise a Thermus thermophilus type II 3-Dehydroquinase polypeptide. In some embodiments, the Thermus thermophilus type II 3-Dehydroquinase polypeptide may comprise an amino acid sequence having at least 70%, at least 80%, at least 85%, at least 90%, at least 95%, at least 97%, at least 98% at least 99% or at least 100% identity with MVLILNGPNLNLLGRREPEVYGRTTLEELEALCEAWGAELGLGVVFRQTNYEGQLIEW VQNAWQEGFLAIVLNPGALTHYSYALLDAIRAQPLPVVEVHLTNLHAREEFRRHSVTAP AARGIVSGFGPLSYKLALVYLAETLEVGGEGF. In some embodiments, the Thermus thermophilus type II 3-Dehydroquinase polypeptide may comprise the amino acid sequence of MVLILNGPNLNLLGRREPEVYGRTTLEELEALCEAWGAELGLGVVFRQTNYEGQLIEW VQNAWQEGFLAIVLNPGALTHYSYALLDAIRAQPLPVVEVHLTNLHAREEFRRHSVTAP AARGIVSGFGPLSYKLALVYLAETLEVGGEGF.

In some embodiments, the 3-Dehydroquinase polypeptide may comprise one or more engineered glycosylation site, wherein the engineered glycosylation site may comprise the amino acid sequence of NXS or NXT, wherein X is not proline. In some embodiments, the one or more engineered glycosylation site is at an amino acid position corresponding to position 1, 25, 32, 49, and/or 63 of the 3-Dehydroquinase polypeptide may comprise the amino acid sequence of NGSVLILNGPNLNLLGRREPEVYGNTTLEELNASAEAWGAELGLGVVFNQTNYEGQLIE WVQNASQEGFLAIVLNPGALTHYSYALLDAIRAQPLPVVEVHLTNLHAREEFRRHSVTA PAARGIVSGFGPLSYKLALVYLAETLEVGGEGF. In some embodiments, the 3-Dehydroquinase polypeptide may comprise 1, 2, 3, 4, or 5 engineered glycosylation sites.

In some embodiments, the 3-Dehydroquinase polypeptide may comprise the amino acid sequence of NGSVLILNGPNLNLLGRREPEVYGNTTLEELNASAEAWGAELGLGVVFNQTNYEGQLIE WVQNASQEGFLAIVLNPGALTHYSYALLDAIRAQPLPVVEVHLTNLHAREEFRRHSVTA PAARGIVSGFGPLSYKLALVYLAETLEVGGEGF.

In some embodiments, a fusion polypeptide described herein may comprise from the N terminus to the C terminus VP-SAD, SAD-VP, VP-SAD-VP, wherein VP and SAD corresponds to the at least one viral polypeptide, and self-assembling domain, respectively.

In some embodiments, the viral polypeptide may comprise the receptor binding domain of the SARS-CoV-2 spike protein which may comprise one or more engineered glycosylation site.

In some embodiments, the VP and SAD are directly linked, wherein VP and SAD corresponds to the at least one viral polypeptide, and self-assembling domain, respectively. In some embodiments, the fusion polypeptide may comprise one or more linkers linking the VP and SAD. In some embodiments, the fusion polypeptide may comprise one or more linkers linking the VP and SAD. In some embodiments, the one or more linker independently may comprise no more than 10 or no more than 5 amino acid residues. In some embodiments, the one or more linker independently may comprise one or more repeats of the GGS or GGGS sequence. In some embodiments, the one or more linker independently may comprise the amino acid sequence of GGS, GGSGGS, GGSGGSGGS, GGGS, GGGSGGGS, or GGGSGGGSGGGS.

In some embodiments, a fusion polypeptide described herein further may comprise an amino acid sequence that targets the fusion polypeptide to the cell surface. In some embodiments, the amino acid sequence that targets the fusion polypeptide to the cell surface may comprise a GPI anchor signal sequence. In some embodiments, the amino acid sequence that targets the fusion polypeptide to the cell surface may comprise a transmembrane domain.

In some embodiments, the fusion polypeptide may comprise an amino acid sequence that targets the fusion polypeptide to the cell surface. In some embodiments, the amino acid sequence that targets the fusion polypeptide to the cell surface may comprise a GPI anchor signal sequence.

In some embodiments, the fusion polypeptide may comprise an amino acid sequence that targets the fusion polypeptide to the cell surface. In some embodiments, the amino acid sequence that targets the fusion polypeptide to the cell surface may comprise a transmembrane domain.

In some embodiments, the transmembrane domain (TM) is from HIV Env of the BG505 isolate (KIFIMIVGGLIGLRIVFAVLSVIHRVR), but any TM domain will suffice. For example, other TM domains could include the TM from SARS-CoV-2 (KWPWYIWLGFIAGLIAIVMVTIML) or the TM from VSV-G (KSSIASFFFIIGLIIGLFLVLR).

In some embodiments, the fusion polypeptide may comprise an amino acid sequence that targets the fusion polypeptide to the cell surface. transmembrane domain may comprise an HIV Env transmembrane domain, a SARS-CoV-2 transmembrane domain, or a VSV-G transmembrane domain. In some embodiments, the HIV Env transmembrane domain may comprise the amino acid sequence of KIFIMIVGGLIGLRIVFAVLSVIHRVR, the SARS-CoV-2 transmembrane domain may comprise the amino acid sequence of KWPWYIWLGFIAGLIAIVMVTIML, and the VSV-G transmembrane domain may comprise the amino acid sequence of KSSIASFFFIIGLIIGLFLVLR.

In some embodiments, the transmembrane domain may comprise a VSV-G transmembrane domain. In some embodiments, the VSV-G transmembrane domain may comprise the amino acid sequence of KSSIASFFFIIGLIIGLFLVLR.

In some embodiments, the viral polypeptide and the transmembrane domain are directly linked. In some embodiments, the viral polypeptide and the transmembrane domain are separated by a linker peptide. In some embodiments, the linker may comprise no more than 10 or no more than 5 amino acid residues. In some embodiments, the linker may comprise one or more repeats of the GGS or GGGS sequence. In some embodiments, the linker may comprise the amino acid sequence of GGS, GGSGGS, GGSGGSGGS, GGGS, GGGSGGGS, or GGGSGGGSGGGS.

In some embodiments, in a fusion polypeptide described herein the viral polypeptide is closer to the N terminus than the transmembrane domain.

In some embodiments, multimerization domains could be added in order to display clusters of the fusion polypeptide on the membrane surface. While tethering to the membrane already should provide a multivalent array of RBDs for B cell interaction, fusion to multimerization domains can enhance the local fusion polypeptide density and concomitantly enhance B cell activation. Such multimerization domains could be added either to a linker, or to the C-terminus of the construct after the TM domain. In some embodiments, the multimerization domain is added after the TM domain. Without being bound by any specific theory, this arrangement would hide the domain from B cell recognition and thus avoid generating non-RBD antibody responses. Examples of small multimerization domains (with fewer than 50 amino acids) include trimerization motifs like the coiled-coil GCN4 (PDB ID: 1GCN) or the trimeric fibritin foldon, or tetramerization motifs like the tetrameric variant of GCN4 in PDB ID 1GCL, or the heptameric coil in PDB ID: 4PNA or the octameric coil in PDB ID: 6G67. Larger multimerization domains with >100 amino acids which include a larger number of CD4 T helper epitopes could also be included. In some embodiments, a lumazine synthase domain that self-assembles into a pentamer can be fused C-terminal to the TM domain, to serve a dual purpose of providing additional T help and providing multimerization. Another example is the protein PH0250 that assembles into a 12-mer ring in PDB ID: 2EKD.

It is noted that these therapeutics may be a chemical compound, a composition which may comprise a polypeptide of the present invention and/or antibody elicited by such a chemical compound and/or portion thereof or a pharmaceutically acceptable salt or a composition which may comprise a polypeptide of the invention, and may be administered alone or as an active ingredient in combination with pharmaceutically acceptable carriers, diluents, and vehicles, as well as other active ingredients.

The compounds or compositions may be administered orally, subcutaneously or parenterally including intravenous, intraarterial, intramuscular, intraperitoneally, and intranasal administration as well as intrathecal and infusion techniques.

It is noted that humans are treated generally longer than the mice or other experimental animals which treatment has a length proportional to the length of the disease process and drug effectiveness. The doses may be single doses or multiple doses over a period of several days, but single doses are preferred. Thus, one may scale up from animal experiments, e.g., rats, mice, and the like, to humans, by techniques from this disclosure and documents cited herein and the knowledge in the art, without undue experimentation.

The treatment generally has a length proportional to the length of the disease process and drug effectiveness and the patient being treated.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier may be a solvent or dispersing medium containing, for example, water, ethanol, polyol (for example, glycerol, propylene glycol, liquid polyethylene glycol, and the like), suitable mixtures thereof, and vegetable oils.

Proper fluidity may be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions.

Additionally, various additives which enhance the stability, sterility, and isotonicity of the compositions, including antimicrobial preservatives, antioxidants, chelating agents, and buffers, may be added. Prevention of the action of microorganisms may be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form may be brought about by the use of agents delaying absorption, for example, aluminum monostearate and gelatin. According to the present invention, however, any vehicle, diluent, or additive used would have to be compatible with the compounds.

Sterile injectable solutions may be prepared by incorporating the compounds utilized in practicing the present invention in the required amount of the appropriate solvent with various amounts of the other ingredients, as desired.

A pharmacological formulation of the present invention, e.g., which may comprise a therapeutic compound or polypeptide of the present invention, may be administered to the patient in an injectable formulation containing any compatible carrier, such as various vehicles, adjuvants, additives, and diluents; or the compounds utilized in the present invention may be administered parenterally to the patient in the form of slow-release subcutaneous implants or targeted delivery systems such as monoclonal antibodies, iontophoretic, polymer matrices, liposomes, and microspheres.

A pharmacological formulation of the compound and composition which may comprise a polypeptide utilized in the present invention may be administered orally to the patient. Conventional methods such as administering the compounds in tablets, suspensions, solutions, emulsions, capsules, powders, syrups and the like are usable. Known techniques, which deliver the compound orally or intravenously and retain the biological activity, are preferred.

In one embodiment, a formulation of the present invention may be administered initially, and thereafter maintained by further administration. For instance, a formulation of the invention may be administered in one type of composition and thereafter further administered in a different or the same type of composition. For example, a formulation of the invention may be administered by intravenous injection to bring blood levels to a suitable level. The patient's levels are then maintained by an oral dosage form, although other forms of administration, dependent upon the patient's condition, may be used. In the instance of a vaccine composition, the vaccine may be administered as a single dose, or the vaccine may incorporate set booster doses. For example, booster doses may comprise variants in order to provide protection against multiple clades of SARS-CoV-2.

The quantity to be administered will vary for the patient being treated and whether the administration is for treatment or prevention and will vary from a few micrograms to a few milligrams for an average 70 kg patient, e.g., 5 micrograms to 5 milligrams such as 500 micrograms, or about 100 ng/kg of body weight to 100 mg/kg of body weight per administration and preferably will be from 10 pg/kg to 10 mg/kg per administration. Typically, however, the antigen is present in an amount on, the order of micrograms to milligrams, or, about 0.001 to about 20 wt %, preferably about 0.01 to about 10 wt %, and most preferably about 0.05 to about 5 wt %.

Of course, for any composition to be administered to an animal or human, including the components thereof, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or RFFIT analysis. Such determinations do not require undue experimentation from the knowledge of the skilled artisan, this disclosure and the documents cited herein. And, the time for sequential administrations may be ascertained without undue experimentation. For instance, dosages may be readily ascertained by those skilled in the art from this disclosure and the knowledge in the art. Thus, the skilled artisan may readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Such determinations do not require undue experimentation from the knowledge of the skilled artisan, this disclosure and the documents cited herein. And, the time for sequential administrations may be ascertained without undue experimentation.

Examples of compositions which may comprise a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions may also be lyophilized. The compositions may contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention may be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers may preferably dispense a metered dose or, a dose having a particular particle size.

Compositions of the invention may contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions may approach solid or gelatin forms, which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, may be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions may be isotonic, i.e., it may have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative may be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems may be readily avoided by reference to standard texts or by simple experiments (not involving undue experimentation), from this disclosure and the documents cited herein.

It is generally envisaged that compounds and compositions of the invention will be administered by injection, as such compounds are to elicit anti-SARS-CoV-2 antibodies, and the skilled artisan may, from this disclosure and the knowledge in the art, formulate compounds and compositions identified by herein methods for administration by injection and administer such compounds and compositions by injection.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions may be administered in dosages and by techniques well known to those skilled in the medical arts taking into consideration such factors as the age, sex, weight, and condition of the particular patient, and the composition form used for administration (e.g., solid vs. liquid). Dosages for humans or other mammals may be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

The present invention will be further illustrated in the following Examples which are given for illustration purposes only and are not intended to limit the invention in any way.

### EXAMPLES

### Example 1

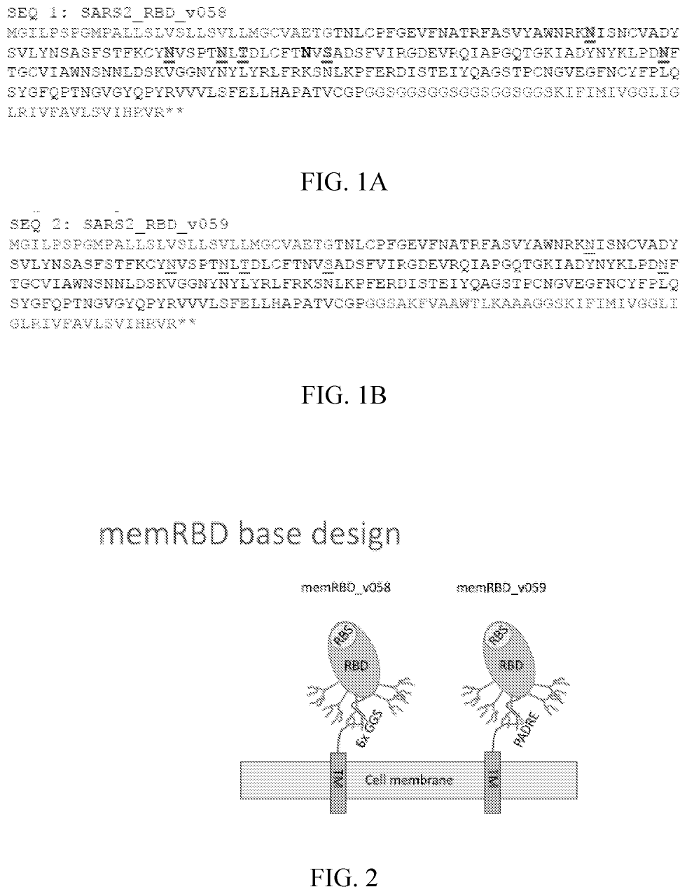

Cell membrane tethered RBDs with glycans. To design the glycan masked receptor binding domain (RBD) the structure of the SARS-CoV-2 spike RBD (PDB ID: 6VSB) was manually inspected for surfaces that would not be accessible to antibody on the trimer but that would be accessible to an antibody on the isolated monomeric RBD. Positions were identified that appeared to be structurally compatible with the presence of an N-linked glycan as well as the mutations required to add a glycosylation motif (NXS/T). This analysis identified eight potential glycosylation sites, at positions 357, 360, 381, 386, 394, 428, 518 and 522 (FIG. 1). Next, a DNA library was assembled encoding RBD variants that contained all combinations of eight glycans (i.e. RBD containing + or − each glycan in all combinations) and that could be displayed on the surface of mammalian cells by tethering to a PDGFR transmembrane domain via a long linker. Using a lentivirus-based method the library was integrated into mammalian 293T cells and the cells were screened using fluorescence activated cell sorting. A key advantage of this method for engineering glycoproteins is that the glycoforms expressed on the glycoproteins will be mammalian. Therefore, expression of the resulting engineered glycoproteins via a nucleic acid or viral vector vaccine delivery modality would be expected to generate glycoproteins with similar properties as discovered during the engineering process. Or, if the glycoproteins are produced as purified protein for vaccine delivery, use of a mammalian cell expression system would also be expected to generate proteins with similar properties as discovered during the engineering process. The library was first sorted using fluorescently labeled human-ACE2 as a selection reagent. Two more rounds of sorting were performed using either human-ACE2 or the non-neutralizing SARS-Cov-2 Ab CR3022 or both as markers for a properly folded RBD. The library from the sorted cells was then isolated and sequenced and it was determined that all eight glycosylation sites were represented in the final sorted sequences (Table 1). This indicated that none of the eight glycosylation sites prevented expression or folding of the RBD, though several of the glycans were underrepresented in the sorted samples compared to what would be expected by chance. One of the recovered sequences contained four glycosylation sites (N357, N381, N394, N428) that appeared to cover much of the RBD buried surface, hence this combination of glycans was initially selected for further testing. One additional glycan (N386) was added to fill in a buried patch that was not covered by the other four glycans. The final design of glycosylation sites in memRBD_v058 and memRBD_v059 contained glycosylation sites at N357, N381, N386, N394, and N428.

To anchor the glycan masked RBD to the cell membrane, the RBDs were fused to the HIV Env transmembrane domain (TM) derived from the BG505 isolate, but truncated the C-terminus of the cytoplasmic domain at a location optimized for maximum expression. Two different linkers connecting the RBD to the TM were tested; a flexible (6×GGS) linker in v058 and a linker containing the PADRE T-cell epitope (AKFVAAWTLKAAA) with GGS on both ends in v059.

Two further rounds of sorting were carried out on the library (sorts 4 and 5) using other SARS-Cov-2 neutralizing and non-neutralizing antibodies as well as ACE2. For sort 4, cells were labeled with the neutralizing antibody CC12.3 as a positive selection reagent. For sort 5, the library was split into 5 separate pools and labeled with the following selection reagents:
Pool 1: ACE2 (positive selection)/CC12.16 (non-neutralizing Ab/negative selection)
Pool 2: ACE2 (positive selection)/CC12.19 (non-neutralizing Ab/negative selection)
Pool 3: CC12.3 (neutralizing Ab/positive selection)
Pool 4: CC6.29 (neutralizing Ab/positive selection)
Pool 5: CC6.30 (neutralizing Ab/positive selection)

The five pools were sorted in parallel, and sequences obtained from the five sorts were enriched more strongly than the earlier sorts for glycosylation sites at positions 357, 394, 518 and 522, suggesting that glycans at those positions may contribute to knocking out binding to non-neutralizing antibodies. Sequences from the final sorts were also strongly under-enriched for glycosylation sites at positions 381 and 386 (Table 1), indicating that glycans at those sites may reduce expression/folding of the RBD when tethered to the membrane via a very long linker and using the PDGFR TM domain. However, memRBD_v058 and memRBD, which include glycosylation sites at both N381 and N386 (but are tethered to a different TM domain via different and shorter linkers), bind very well to all neutralizing antibodies tested, demonstraing that the presence of N381 and N386 glycosylation sites is compatible with good expression and proper folding of the RBD.

Among the 55 full-length high-quality sequences isolated from sorting experiments that required binding to a conformational antibody (neutralizing or non-neutralizing) or the receptor ACE2, 35 different combinations of the eight glycosylation sites were observed in our original library, meaning that 64% of the sequences represented unique combinations of the glycosylation sites in our library (Table 2). Furthermore, every glycosylation site was observed in the library at least six times, meaning that each glycosylation site was observed in at least 9% of all sequences (Table 3). Based on those observations, and based on the enrichments of individual sites (Table 1), we conclude that the majority of all possible combinations of the glycosylation sites in the library are compatible with RBD folding and binding to conformation-specific and/or neutralizing antibodies. The different combinations will inhibit the binding of non-neutralizing antibodies to varying degrees. By virtue of the locations of these glycosylation sites (at positions expected to be buried or not well-exposed on the intact spike), any combination of these glycosylation sites would inhibit the binding of some non-neutralizing antibodies. Features and characteristics of exemplary combinations are outlined in Table 4.

Table 1 shows the frequency of glycosylation sites in sequences isolated after sorting with the indicated selection reagents. (+), cells showing the highest fluorescence intensity for the indicated selection reagent were sorted. (−), cells showing the lowest fluorescence intensity for the indicated selection reagent were sorted. The frequency of each glycosylation site is normalized to the expected theoretical frequency (i.e. frequencies >1 are positively enriched and frequencies <1 are negatively enriched).

TABLE 1
Frequency of glycosylation sites in sequences isolated after sorting with the indicated selection reagents.

| | number of sorts | | | | | | | | |
|---|---|---|---|---|---|---|---|---|---|
| | 2 | 3 | 3 | 3 | 5 | 5 | 5 | 5 | 5 |
| | (+ or −) sort reagent | | | | | | | | |
| | | | | ACE2(+) | ACE2(+) | ACE2(+) | | | |
| | CR3022 (+) | ACE2 (+) | CR3022 (+) | CR3022(+) | CC12.16(−) | CC12.19(−) | CC12.3(+) | CC6.29(+) | CC6.30(+) |
| N357 | 1.85 | 0.76 | 1.75 | 1.17 | 2.40 | 2.29 | 0.67 | 0.57 | 0.80 |
| N360 | 0.62 | 1.33 | 0.75 | 1.67 | 0.00 | 1.14 | 0.67 | 2.86 | 1.20 |
| N381 | 0.92 | 1.52 | 0.50 | 0.83 | 0.00 | 0.00 | 0.00 | 0.00 | 0.40 |
| N386 | 0.92 | 1.71 | 0.25 | 0.17 | 0.80 | 0.00 | 0.00 | 0.00 | 0.40 |
| N394 | 0.92 | 0.19 | 0.63 | 0.58 | 1.60 | 0.86 | 0.67 | 0.29 | 0.60 |
| N428 | 1.23 | 0.76 | 0.63 | 0.92 | 1.60 | 0.57 | 1.67 | 0.86 | 1.40 |
| N518 | 1.08 | 0.77 | 1.17 | 1.00 | 2.00 | 1.14 | 1.00 | 1.14 | 1.20 |
| N522 | 0.00 | 1.28 | 2.78 | 2.08 | 6.67 | 2.38 | 0.00 | 7.14 | 1.67 |

TABLE 2
Listing of all unique combinations of glycosylation
sites observed in full-length high quality sequences
in our library sorting experiments

| | |
|---|---|
| 1 | ---- ---- ---- ---- ---- ---- ---- ---- |
| 2 | ---- ---- ---- ---- ---- ---- N518 ---- |
| 3 | ---- ---- ---- ---- ---- ---- N518 N522 |
| 4 | ---- ---- ---- ---- ---- N428 ---- ---- |
| 5 | ---- ---- ---- ---- ---- N428 N518 ---- |
| 6 | ---- ---- ---- ---- ---- N428 N518 N522 |
| 7 | ---- ---- ---- ---- N394 ---- ---- ---- |
| 8 | ---- ---- ---- ---- N394 ---- N518 ---- |
| 9 | ---- ---- ---- ---- N394 N428 ---- ---- |
| 10 | ---- ---- ---- ---- N394 N428 N518 N522 |
| 11 | ---- ---- ---- N386 N394 ---- N518 N522 |
| 12 | ---- ---- N381 ---- ---- ---- N518 ---- |
| 13 | ---- N360 ---- ---- ---- ---- ---- ---- |
| 14 | ---- N360 ---- ---- ---- ---- ---- N522 |
| 15 | ---- N360 ---- ---- ---- ---- N518 ---- |
| 16 | ---- N360 ---- ---- ---- N428 ---- ---- |
| 17 | ---- N360 ---- ---- ---- N428 N518 ---- |
| 18 | ---- N360 ---- ---- ---- N428 N518 N522 |
| 19 | ---- N360 ---- ---- N394 N428 N518 ---- |
| 20 | ---- N360 ---- ---- N394 N428 N518 N522 |
| 21 | ---- N360 ---- N386 ---- N428 ---- ---- |
| 22 | ---- N360 N381 ---- N394 ---- N518 ---- |
| 23 | ---- N360 N381 N386 ---- N428 N518 ---- |
| 24 | N357 ---- ---- ---- ---- ---- ---- ---- |
| 25 | N357 ---- ---- ---- ---- ---- N518 ---- |
| 26 | N357 ---- ---- ---- ---- N428 N518 ---- |
| 27 | N357 ---- ---- ---- N394 ---- ---- ---- |
| 28 | N357 ---- ---- ---- N394 N428 N518 ---- |
| 29 | N357 ---- N381 ---- N394 N428 ---- ---- |
| 30 | N357 N360 ---- ---- ---- ---- ---- ---- |
| 31 | N357 N360 ---- ---- ---- ---- N518 N522 |
| 32 | N357 N360 ---- ---- ---- N428 ---- ---- |
| 33 | N357 N360 ---- ---- ---- N428 N518 ---- |
| 34 | N357 N360 ---- ---- N394 ---- N518 ---- |
| 35 | N357 N360 ---- N386 N394 N428 ---- ---- |

TABLE 3
Number of observations and overall frequency for each
glycosylation site, among all 59 full-length high quality
sequences recovered from sorting experiments

| Glycosylation | No. of | Overall |
|---|---|---|
| site | observations | Frequency |
| N357 | 20 | 0.36 |
| N360 | 20 | 0.36 |
| N381 | 5 | 0.09 |
| N386 | 5 | 0.09 |
| N394 | 19 | 0.35 |
| N428 | 31 | 0.56 |
| N518 | 32 | 0.58 |
| N522 | 9 | 0.16 |

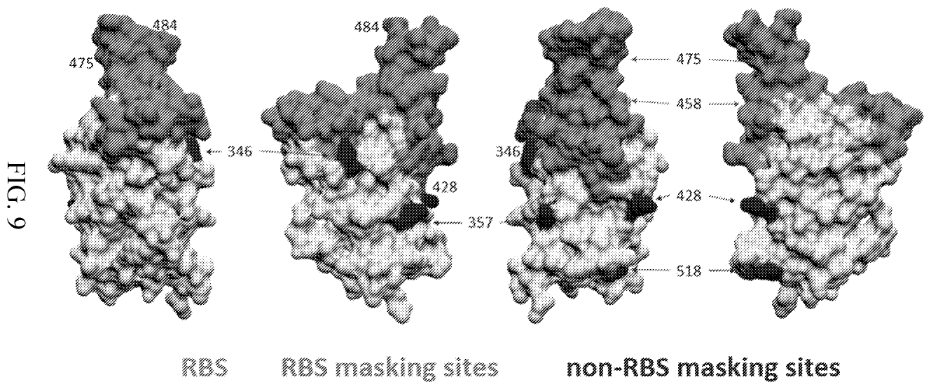

TABLE 4
Exemplary combinations based on antigenic profile

| Combination | 357 | 381 | 386 | 394 | 428 | 518 | 522 | Comment |
|---|---|---|---|---|---|---|---|---|
| 1 | x | x | x | x | x | | | v058/v059, antigenic |
| | | | | | | | | profile in FIG. 4 |
| 2 | x | | | x | x | x | x | most enriched |
| | | | | | | | | individual positions |
| 3 | x | | | x | x | x | | most frequent |
| | | | | | | | | combination observed |
| 4 | x | | x | x | x | x | | most frequent + 386 |
| | | | | | | | | from v058/059 |
| 5 | | | x | x | | x | x | recovered seq. w/fewer |
| | | | | | | | | glycans but still good |
| | | | | | | | | surface coverage |
| 6 | x | x | x | x | x | x | x | v058/v059 + highly |
| | | | | | | | | enriched 518/522 |
| 7 | x | | x | x | x | x | x | most frequent + |
| | | | | | | | | 386(v058/v059) + |
| | | | | | | | | 522(enriched) |
| 8 | x | x | | x | | x | | g3.6, higher expression |
| | | | | | | | | than v058/v059 but |
| | | | | | | | | retains good antigenic |
| | | | | | | | | profile (FIG. 8) |
| 9 | x | x | | x | x | | | g3.7, good antigenic |
| | | | | | | | | profile (FIG. 8) |

### Example 2

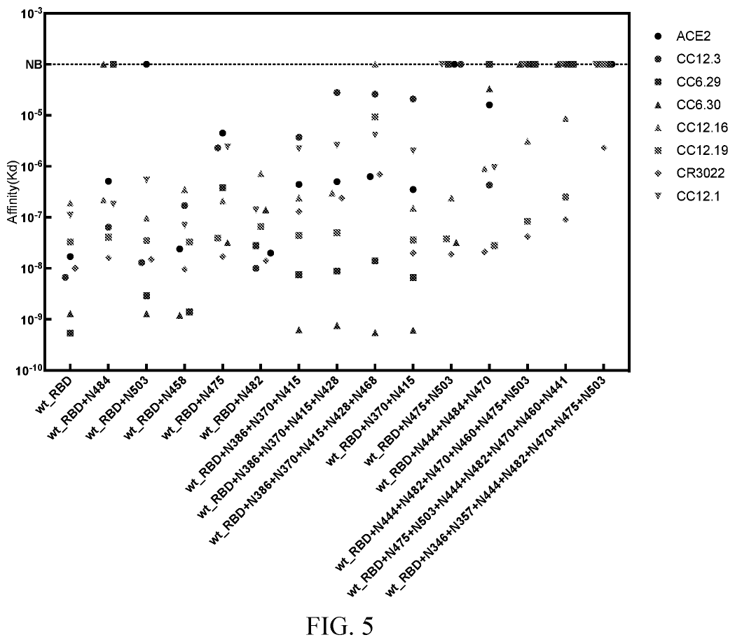

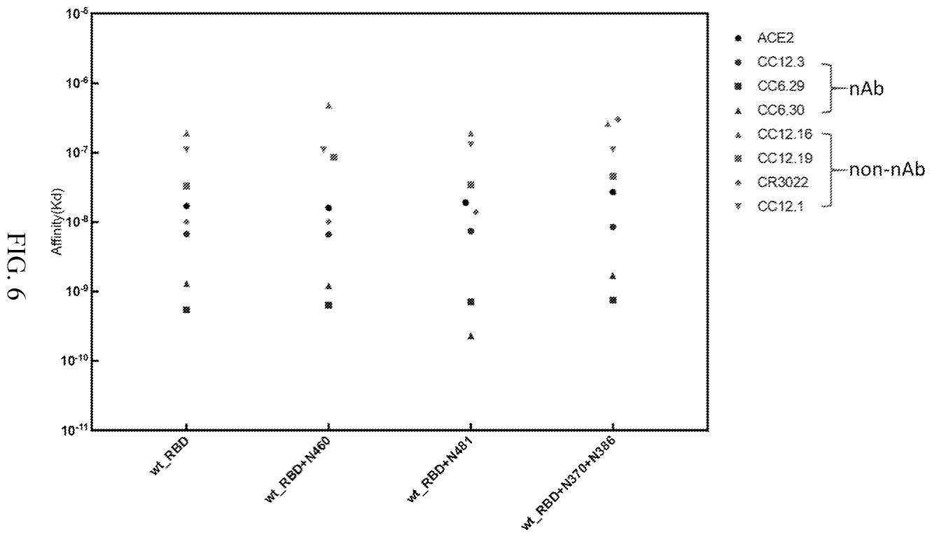

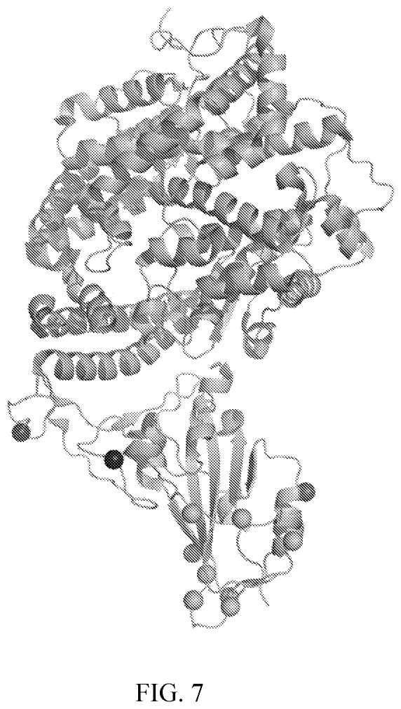

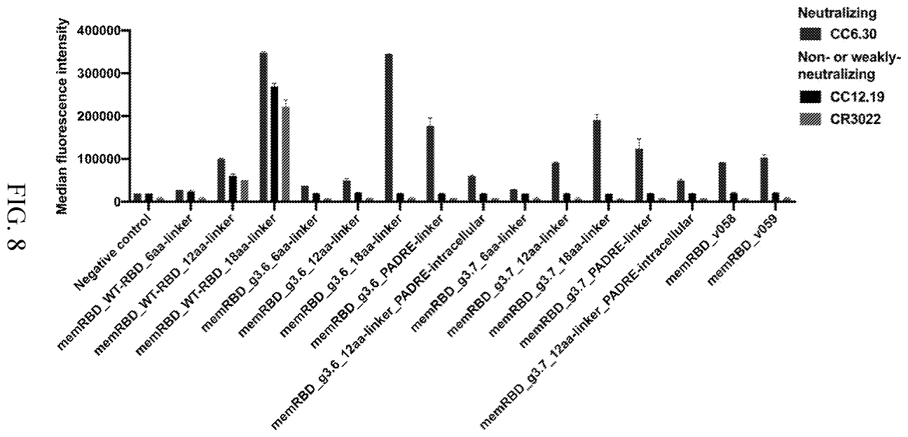

Soluble RBDs with glycans. In addition to our screening of membrane-tethered RBDs with engineered glycoslation sites, soluble RBDs with engineered glycosylation sites were tested. In this case, 293 cells were transfected with DNA encoding RBDs without a tether or TM domain, and the RBDs were purified by Ni+ affinity-chromatography and size exclusion chromatography. Antibodies were captured on an SPR instrument sensor surface and RBDs were flowed as analytes. A variety of single and double glycosylation site combinations were tested. Multiple positions and combinations of positions were found at which addition of a glycosylation site causes significant reduction of binding of nAbs or ACE2 (FIG. 5), further that glycosylation sites can be added at positions 460 and 481 without substantial reduction of ACE2 or nAb binding (FIG. 6). These two glycosylation sites are located within the loop region (454-492) underneath the ACE2 binding site (blue spheres in FIG. 7). Glycans at these sites would not be expected to interfere with any Ab that binds directly to the ACE2 binding site, where most of the potent nAbs bind. Accordingly, adding glycans to these sites can help to focus responses toward the ACE2 binding site where the majority of the most potent nAbs bind.

Analysis of soluble RBDs with engineered glycosylation sites also showed that adding glycosylation sites at N386 and N370 generally does not affect binding of nAbs but does significantly reduce binding by the non-nAb CR3022 (FIG. 6). This finding further supports inclusion of the N386 glycan in certain glycan combinations. Position 370 is in the region of the RBD distal from the ACE2 binding site (FIG. 7), hence glycans at this position would not be expected to interfere with binding of potent nAbs. Thus, favorable antigenicity is preserved in the presence of glycosylation sites at 370 as well as 386.

Glycan masked RBDs could also have utility as diagnostic serological probes. For example, an RBD with glycans masking all or most non-nAb binding sites would be optimal for detection of nAbs in serum binding assays. On the other hand, glycan-masked RBDs with glycans masking nAb binding sites but leaving non-nAb binding sites exposed, would be optimal for detection of RBD-directed non-nAbs in serum binding assays. Accordingly, the invention provides engineered RBDs as serological probes with all combinations of the glycosylation sites disclosed herein, individually or in combinations.

Our analysis of soluble RBDs with engineered glycosylation sites also showed that adding a glycosylation site at N503 caused a precision knockout of ACE2 binding. ACE2 binding was reduced to undetectable while binding of other nAbs and non-nAbs was altered by less than a factor of 10 in all cases (FIG. 5). This serves as an example for how engineered glycosylation sites can offer tailored specificity in RBD serological probes.

The glycosylation site at 503 is also useful in vaccine formulations, in which preventing ACE2 binding would prevent immunogens from interacting with ACE2 in vivo and hence would ensure that the ACE2 binding site remains exposed for elicitation of ACE2-binding-site-directed nAbs. Accordingly the invention includes RBDs with a glycosylation site added to position 503.

### Example 3

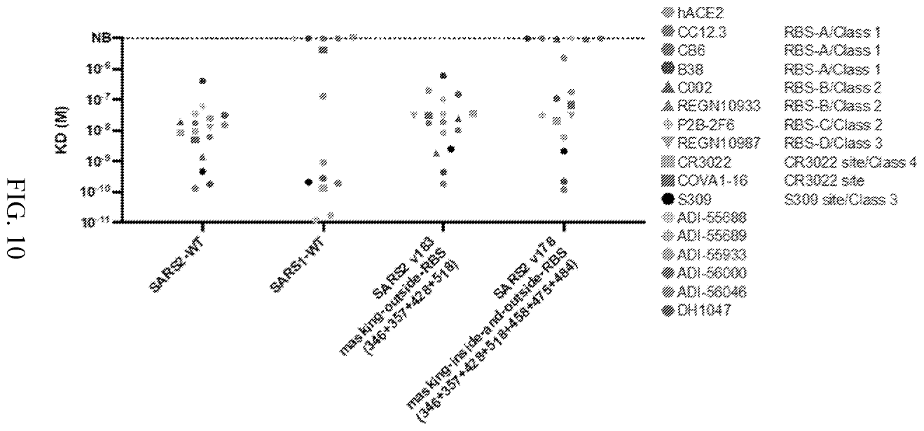

FIG. 9 depicts locations of glycan masking sites on SARS-CoV-2 RBD. Locations of glycan masking sites on SARS-CoV-2 RBD. Positions are indicated for engineered glycosylation sites for masking the RBS and sites outside the RBS. The SARS-CoV-2 RBD is shown in surface representation in four different orientations, with each panel differing from its neighbors by 900 rotation about a vertical axis. Surface coloring as follows: RBS, green; RBS masking glycosylation sites, purple; glycosylation sites masking non-RBS epitopes, blue.

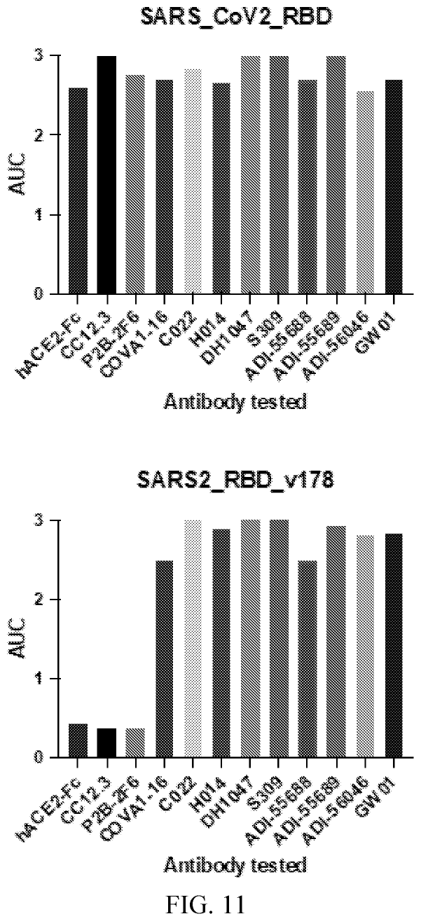

Information on hACE2 and antibodies in the SPR or ELISA data in FIGS. 10 and 11 is presented into the table below:

| | | Epitope | |
|---|---|---|---|
| | | within | |
| Antibody | type of Ab | RBD | Reference |
| hACE2-Fc | dimeric form | RBS | |
| | of human | | |
| | ACE2 | | |
| | receptor | | |
| CC12.3 | specific for | RBS-A/ | Rogers et al Science |
| | SARS-CoV-2 | class 1 | 2020; PMID: 32540903 |
| CB6 | specific for | RBS-A/ | Shi et al. Nature 2020; |
| | SARS-CoV-2 | class 1 | PMID: 32454512 |
| B38 | specific for | RBS-A/ | Wu et al. Science 2020; |
| | SARS-CoV-2 | class 1 | PMID: 32404477 |
| P2B-2F6 | specific for | RBS-C/ | Ju et al. Nature 2020; |
| | SARS-CoV-2 | class 2 | PMID: 32454513 |
| REGN10987 | specific for | RBS-D/ | Hansen et al. Science |
| | SARS-CoV-2 | class 3 | 2020; PMID: 32540901 |
| CR3022 | weakly cross- | CR3022 | Yuan et al. Science |
| | neutralizing | site | 2020; PMID: 32245784 |
| COVA1-16 | cross- | CR3022 | Brouwer et al. Science |
| | neutralizing to | site | 2020; PMID: 32540902 |
| | diverse | | |
| | sarbecoviruses | | |
| C022 | cross- | CR3022 | Robbiani et al Nature |
| | neutralizing to | site | 2020; PMID: 32555388 |
| | diverse | (likely) | |
| | sarbecoviruses | | |
| H014 | cross- | overlaps | Lv et al. Science |
| | neutralizing to | CR3022, | 2020; PMID: 32703908 |
| | diverse | but | |
| | sarbecoviruses | closer to | |
| | | RBS | |
| DH1047 | cross- | overlaps | Martinez et al BioRxiv; |
| | neutralizing to | CR3022, | PMID: 33948590 |
| | diverse | but | |
| | sarbecoviruses | closer to | |
| | | RBS | |
| S309 | cross- | S309 | Pinto et al Nature |
| | neutralizing to | site/ | 2020; PMID: 32422645 |
| | diverse | class 3 | |
| | sarbecoviruses | | |
| ADI-56888 | cross- | | Wec et al Science |
| | neutralizing to | | 2020; PMID: 32540900 |
| | diverse | | |
| | sarbecoviruses | | |
| ADI-55689 | cross- | | Wec et al Science |
| | neutralizing to | | 2020; PMID: 32540900 |
| | diverse | | |
| | sarbecoviruses | | |
| ADI-55933 | cross- | | Wec et al Science |
| | neutralizing to | | 2020; PMID: 32540900 |
| | diverse | | |
| | sarbecoviruses | | |
| ADI-56000 | cross- | | Wec et al Science |
| | neutralizing to | | 2020; PMID: 32540900 |
| | diverse | | |
| | sarbecoviruses | | |
| ADI-56046 | cross- | | Wec et al Science |
| | neutralizing to | | 2020; PMID: 32540900 |
| | diverse | | |
| | sarbecoviruses | | |
| GW01 | cross- | | CN111793129A |
| | neutralizing to | | |
| | diverse | | |
| | sarbecoviruses | | |

FIG. 10 depicts glycan masking of non-conserved or non-neutralizing epitopes in SARS-CoV-2 RBD, including masking of the non-conserved immunodominant receptor binding site (RBS). Dissociation constants measured by SPR for hACE2 and the indicated antibodies binding to wild-type (WT) and engineered receptor binding domains (RBDs). SARS2-WT, wild-type RBD for SARS-CoV-2; SARS1-WT, wild-type RBD for SARS-CoV; SARS2 v183, glycan-masked RBD for SARS-CoV-2 with glycans added at the indicated positions to mask non-conserved or non-neutralizing sites outside the RBS; SARS2 v178, glycan-masked RBD for SARS-CoV-2 with glycans added at the indicated positions to mask the RBS and to mask non-conserved or non-neutralizing sites outside the RBS. The data show that glycan masking eliminates or weakens binding of hACE2 and RBS-specific antibodies but maintains high-affinity binding to cross-neutralizing antibodies. The key shows epitope classification obtained from published literature as: “RBS-X” from M. Yuan et al., Science 10.1126/science.abh1139 (2021) and “Class X” from Barnes et al., Nature 2020 https://doi.org/10.1038/s41586-020-2852-1. The data proves that Applicants' v178 design with RBS masking knocks out binding to all classes of RBS-specific antibodies, while maintaining high affinity binding to cross-neutralizing antibodies. This feature may be important for designing vaccines to elicit broadly neutralizing antibodies against sarbecoviruses.

FIG. 11 depicts SARS-CoV-2 RBD with 0 glycosylation sites added (top) and SARS-CoV-2 RBD with 7 glycosylation sites added: 3 to mask RBS (458, 475, 484) and 4 to mask non-conserved or non-neutralizing epitopes (346+357+428+518) (bottom). ELISA measurement of antibody binding to WT SARS-CoV-2 RBD and glycan-masked SARS-CoV-2 RBD. RBDs were captured on the ELISA plate. hACE2-Fc or the indicated IgGs were titrated for binding, and the area under the response curve (AUC) was calculated in Prism. SARS_CoV2_RBD, wild-type RBD for SARS-CoV-2; SARS2_RBD_v178, glycan-masked RBD for SARS-CoV-2 with glycans added to mask the RBS and to mask non-conserved or non-neutralizing sites outside the RBS. The data show that glycan masking effectively eliminates or greatly weakens binding of hACE2 and RBS-specific antibodies but maintains high avidity binding to cross-neutralizing antibodies.

Although the present invention and its advantages have been described in detail, it should be understood that various changes, substitutions and alterations can be made herein without departing from the spirit and scope of the invention as defined in the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A non-naturally occurring pathogen surface glycoprotein receptor binding domain (RBD) comprising an engineered glycosylation site.

2. The non-naturally occurring pathogen surface glycoprotein RBD of claim 1 wherein the engineered glycosylation site comprises substitution of N at the position to be glycosylated or substitution of S or T at the position two amino acids towards the C-terminus from an existing N of the surface glycoprotein RBD, or substitution of N at the position to be glycosylated and substitution of S or T at the position two amino acids towards the C-terminus from the substituted N of the surface glycoprotein RBD so as to create the motif N-X-S/T, so long as X is not proline.

3. The non-naturally occurring pathogen surface glycoprotein RBD of claim 1 wherein the pathogen is a coronavirus.

4. The non-naturally occurring pathogen surface glycoprotein RBD of claim 3 comprising a sequence that is at least 70%, at least 80%, at least 85%, at least 90%, at least 95%, at least 96%, at least 97%, at least 98%, or at least 99%, identical to the RBD of the SARS-CoV-2-S surface glycoprotein; or comprising a sequence that is at least 70%, at least 80%, at least 85%, at least 90%, at least 95%, at least 96%, at least 97%, at least 98%, or at least 99%, identical to the RBD of the SARS-CoV-S or SARS-CoV-2-S.

5. The non-naturally occurring pathogen surface glycoprotein RBD of claim 3 comprising an engineered glycosylation site at one or more of amino acid positions 357, 360, 370, 381, 386, 394, 428, 460, 481, 503, 518, and 522, according to the amino acid numbering of the SARS-CoV-2-S surface glycoprotein.

6. The non-naturally occurring pathogen surface glycoprotein RBD of claim 5 comprising, with reference to the amino acid numbering of the SARS-CoV-2-S surface glycoprotein, an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428, or at positions 357, 381, 394 and 518 positions 346, 357, 428, 458, 518, 484, and 475 or at positions 346, 357, 428, or 518.

7. The non-naturally occurring pathogen surface glycoprotein RBD of claim 3 comprising, with reference to the amino acid numbering of the SARS-CoV-2-S surface glycoprotein, an engineered N-glycosylation site at one or two or three or four or five or six or seven or eight of positions 346, 357, 360, 370, 381, 386, 394, 428, 458, 460, 475, 481, 484, 503, 518, and 522 in any combination.

8. The non-naturally occurring pathogen surface glycoprotein RBD of claim 7, which comprises an engineered N-glycosylation site at positions 357, 381, 386, 394, and 528 (SEQ ID NO:13), or at positions 357, 394, 428, 518, and 522 (SEQ ID NO:14), or at positions 357, 394, 428, and 518 (SEQ ID NO:15), or at positions 357, 386, 394, 428, and 518 (SEQ ID NO:16), or at positions 386, 394, 518, and 522 (SEQ ID NO:17), or at positions 357, 381, 386, 394, 428, 518, and 522 (SEQ ID NO:18), or at positions 357, 386, 394, 428, 518, and 522 (SEQ ID NO:19), or at a self-assembling domain capable of forming a nanoparticle, prefusion stabilized membrane-anchored SARS-CoV-2 full-length spike protein, or at positions 357, 381, 394, and 518 (SEQ ID NO:20) positions 346, 357, 428, 458, 518, 484, and 475 (SEQ ID NO: 23) or at positions 346, 357, 428, or 518 (SEQ ID NO: 24).

9. The non-naturally occurring pathogen surface glycoprotein RBD of claim 7, comprising glycosylation sites in a combination selected from Table 2.

10. The non-naturally occurring pathogen surface glycoprotein RBD of claim 5, further comprising an N-glycosylation site at one or both of positions 460 and 481.

11. The non-naturally occurring pathogen surface glycoprotein RBD of claim 5, further comprising an N-glycosylation site at one or both of positions 370 and 386.

12. The non-naturally occurring pathogen surface glycoprotein RBD of claim 1 wherein the RBD includes a linker to a transmembrane domain of the pathogen or coronavirus surface glycoprotein for cell surface expression.

13. The non-naturally occurring pathogen surface glycoprotein RBD of claim 12 wherein the linker comprises a glycine rich linker, or GGSGGSGGSGGSGGS (SEQ ID NO: 34), or a T-cell epitope, or a PADRE CD4 T cell epitope.

14. A non-naturally occurring pathogen surface glycoprotein comprising the non-naturally occurring pathogen surface glycoprotein RBD of claim 1.

15. The non-naturally occurring pathogen of claim 14 comprising a secretion signal sequence, prefusion stabilized membrane-anchored SARS-CoV-2 full-length spike protein, and/or an amino acid sequence that targets the fusion polypeptide to the cell surface comprising a GPI anchor signal sequence.

16. The non-naturally occurring pathogen or coronavirus surface glycoprotein of claim 14 including a moiety capable of binding to a metal hydroxide adjuvant; a moiety capable of binding to a metal hydroxide adjuvant at or near comprising within 25 amino acids of the N- or C-terminus; a moiety capable of binding to a metal hydroxide adjuvant comprising phosphoserine; a moiety capable of binding to a metal hydroxide adjuvant at or near comprising within 25 amino acids of the N- or C-terminus comprising phosphoserine; a moiety capable of binding to a metal hydroxide adjuvant comprising cysteine; a moiety capable of binding to a metal hydroxide adjuvant at or near comprising within 25 amino acids of the N- or C-terminus comprising cysteine; or any of the foregoing wherein the metal hydroxide adjuvant comprises aluminum hydroxide or alum or sodium bis(2-methoxyethoxy)aluminum hydride; or any of the foregoing comprising phosphoserine that can couple with a cysteine.

17. A non-naturally occurring nucleic acid molecule encoding the non-naturally occurring pathogen surface glycoprotein RBD of claim 1.

18. A vector comprising a regulatory element operable in a eukaryotic cell operably linked to the nucleic acid of claim 17.

19-25. (canceled)

26. An immunogenic or vaccine composition comprising a pharmaceutically or veterinarily acceptable carrier and an effective amount to elicit an immune response, or an effective amount to elicit a protective immune response, of: the non-naturally occurring pathogen surface glycoprotein RBD of claim 1.

27-31. (canceled)

32. A method for eliciting an immune or protective immune response in a mammal, or for eliciting, stimulating or producing an antibody or antibody response in a mammal, or for eliciting, stimulating or producing a neutralizing antibody (nAb) response in a mammal comprising administering an effective amount of the non-naturally occurring pathogen surface glycoprotein RBD of claim 1.

33-34. (canceled)

35. A serological probe capable of detecting an antibody in serum or other antibody mixture which comprises an engineered glycosylation site at one or more of amino acid positions 357, 360, 370, 381, 386, 394, 428, 460, 481, 503, 518, and 522, according to the amino acid numbering of the SARS-CoV-2-S surface glycoprotein.

36-37. (canceled)


SEQUENCE LISTING (43 sequences)

SEQ ID NO: 1 - protein, 271 aa, Artificial Sequence
  Description of Artificial Sequence Synthetic polypeptide
MGILPSPGMPALLSLVSLLSVLLMGCVAETGTNLCPFGEVFNATRFASVYAWNRKNISNCVADYSVLYNSASFSTFKCYNVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSGGSGGSGGSGGSGGSKIFIMIVGGLIGLRIVFAVLSVIHRVR

SEQ ID NO: 2 - protein, 272 aa, Artificial Sequence
  Description of Artificial Sequence Synthetic polypeptide
MGILPSPGMPALLSLVSLLSVLLMGCVAETGTNLCPFGEVFNATRFASVYAWNRKNISNCVADYSVLYNSASFSTFKCYNVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSAKFVAAWTLKAAAGGSKIFIMIVGGLIGLRIVFAVLSVIHRVR

SEQ ID NO: 3 - protein, 18 aa, Artificial Sequence
  Description of Artificial Sequence Synthetic peptide
GGSGGSGGSGGSGGSGGS

SEQ ID NO: 4 - protein, 19 aa, Artificial Sequence
  Description of Artificial Sequence Synthetic peptide
GGSAKFVAAWTLKAAAGGS

SEQ ID NO: 5 - protein, 31 aa, Artificial Sequence
  Description of Artificial Sequence Synthetic polypeptide
MGILPSPGMPALLSLVSLLSVLLMGCVAETG

SEQ ID NO: 6 - protein, 27 aa, Human immunodeficiency virus
KIFIMIVGGLIGLRIVFAVLSVIHRVR

SEQ ID NO: 7 - protein, 24 aa, Severe acute respiratory syndrome coronavirus 2
KWPWYIWLGFIAGLIAIVMVTIML

SEQ ID NO: 8 - protein, 22 aa, Vesicular stomatitis virus
KSSIASFFFIIGLIIGLFLVLR

SEQ ID NO: 9 - protein, 195 aa, Severe acute respiratory syndrome coronavirus 2 [sequence omitted]

SEQ ID NO: 10 - protein, 194 aa, Severe acute respiratory syndrome-related coronavirus
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGP

SEQ ID NO: 11 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 13 - protein, 195 aa, Artificial Sequence
  Description of Artificial Sequence Synthetic polypeptide
  MOD_RES (56)..(56): S or T
TNLCPFGEVFNATRFASVYAWNRKNISNCVADYSVLYNSASFSTFKCYNVSPTNLXDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 14 - protein, 195 aa, Artificial Sequence
  Description of Artificial Sequence Synthetic polypeptide
  MOD_RES (188)..(188): S or T
  MOD_RES (192)..(192): S or T
TNLCPFGEVFNATRFASVYAWNRKNISNCVADYSVLYNSASFSTFKCYGVSPTKLNDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 15 - protein, 195 aa, Artificial Sequence
  Description of Artificial Sequence Synthetic polypeptide
  MOD_RES (64)..(64): S or T
  MOD_RES (188)..(188): S or T
TNLCPFGEVFNATRFASVYAWNRKNISNCVADYSVLYNSASFSTFKCYGVSPTKLNDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 16 - protein, 195 aa, Artificial Sequence
  Description of Artificial Sequence Synthetic polypeptide
  MOD_RES (56)..(56): S or T
  MOD_RES (64)..(64): S or T
  MOD_RES (188)..(188): S or T
TNLCPFGEVFNATRFASVYAWNRKNISNCVADYSVLYNSASFSTFKCYGVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 17 - protein, 195 aa, Artificial Sequence
  Description of Artificial Sequence Synthetic polypeptide
  MOD_RES (56)..(56): S or T
  MOD_RES (64)..(64): S or T
  MOD_RES (188)..(188): S or T
  MOD_RES (192)..(192): S or T
TNLCPFGEVFNATRFASVYAWNRKRISNCVADYSVLYNSASFSTFKCYGVSPTNLXDLCFTNVXADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 18 - protein, 195 aa, Artificial Sequence
  Description of Artificial Sequence Synthetic polypeptide
  MOD_RES (56)..(56): S or T
  MOD_RES (64)..(64): S or T
  MOD_RES (188)..(188): S or T
  MOD_RES (192)..(192): S or T
TNLCPFGEVFNATRFASVYAWNRKNISNCVADYSVLYNSASFSTFKCYNVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 19 - protein, 195 aa, Artificial Sequence
  Description of Artificial Sequence Synthetic polypeptide
  MOD_RES (56)..(56): S or T
  MOD_RES (64)..(64): S or T
  MOD_RES (188)..(188): S or T
  MOD_RES (192)..(192): S or T
TNLCPFGEVFNATRFASVYAWNRKNISNCVADYSVLYNSASFSTFKCYGVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 20 - protein, 195 aa, Artificial Sequence
  Description of Artificial Sequence Synthetic polypeptide
  MOD_RES (64)..(64): S or T
  MOD_RES (188)..(188): S or T
TNLCPFGEVFNATRFASVYAWNRKNISNCVADYSVLYNSASFSTFKCYNVSPTKLNDLCFTNVXADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 21 - protein, 195 aa, Artificial Sequence
  Description of Artificial Sequence Synthetic polypeptide
  MOD_RES (64)..(64): S or T
TNLCPFGEVFNATRFASVYAWNRKNISNCVADYSVLYNSASFSTFKCYNVSPTKLNDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 22 - protein, 195 aa, Artificial Sequence
  Description of Artificial Sequence Synthetic polypeptide
  MOD_RES (30)..(30): S or T
  MOD_RES (40)..(40): S or T
  MOD_RES (56)..(56): S or T
  MOD_RES (64)..(64): S or T
  MOD_RES (130)..(130): S or T
  MOD_RES (151)..(151): S or T
  MOD_RES (173)..(173): S or T
  MOD_RES (188)..(188): S or T
  MOD_RES (192)..(192): S or T
TNLCPFGEVFNATRFASVYAWNRKNISNCXADYSVLYNSXSFSTFKCYNVSPTNLXDLCFTNVXADSFVIRGDEVRQIAPGQTGKIADYNYKLPDNFTGCVIAWNSNNLDSKVGGNYNYLYRLFRKSNLXPFERDISTEIYQAGSTPCNGXEGFNCYFPLQSYGFQPTNGNGXQPYRVVVLSFELNHXPNTXCGP

SEQ ID NO: 23 - protein, 197 aa, Artificial Sequence
  Description of Artificial Sequence Synthetic polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRNSTLKPFERDISTEIYQNGSTPCNGVNGTNCYFPLQSYGFQPTNGVGYQPYRVVVLSFELNHTPATVCGP

SEQ ID NO: 24 - protein, 197 aa, Artificial Sequence
  Description of Artificial Sequence Synthetic polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGP

SEQ ID NO: 25 - protein, 18 aa, Clostridium tetani
NILMQYIKANSKFIGITE

SEQ ID NO: 26 - protein, 16 aa, Clostridium tetani
LKFIIKRYTPNNEIDS

SEQ ID NO: 27 - protein, 11 aa, Clostridium tetani
LYNGLKFIIKR

SEQ ID NO: 28 - protein, 20 aa, Clostridium tetani
LYNGLKFIIKRYTPNNEIDS

SEQ ID NO: 29 - protein, 14 aa, Corynebacterium diphtheriae
KTTAALSILPGIGS

SEQ ID NO: 30 - protein, 15 aa, Hepatitis B virus
FFLLTRILTIPQSLD

SEQ ID NO: 31 - protein, 19 aa, Plasmodium falciparum
EYLNKIQNSLSTEWSCSVT

SEQ ID NO: 32 - protein, 14 aa, Clostridium tetani
QYIKANSKFIGITE

SEQ ID NO: 33 - protein, 12 aa, Clostridium tetani
NILMQYIKANSK

SEQ ID NO: 34 - protein, 15 aa, Artificial Sequence
  Description of Artificial Sequence Synthetic peptide
GGSGGSGGSGGSGGS

SEQ ID NO: 35 - protein, 149 aa, Thermus thermophilus
MVLILNGPNLNLLGRREPEVYGRTTLEELEALCEAWGAELGLGVVFRQTNYEGQLIEWVQNAWQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 36 - protein, 151 aa, Artificial Sequence
  Description of Artificial Sequence Synthetic polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 37 - protein, 4 aa, Artificial Sequence
  Description of Artificial Sequence Synthetic peptide
GGGS

SEQ ID NO: 38 - protein, 6 aa, Artificial Sequence
  Description of Artificial Sequence Synthetic peptide
GGSGGS

SEQ ID NO: 39 - protein, 9 aa, Artificial Sequence
  Description of Artificial Sequence Synthetic peptide
GGSGGSGGS

SEQ ID NO: 40 - protein, 8 aa, Artificial Sequence
  Description of Artificial Sequence Synthetic peptide
GGGSGGGS

SEQ ID NO: 41 - protein, 12 aa, Artificial Sequence
  Description of Artificial Sequence Synthetic peptide
GGGSGGGSGGGS

SEQ ID NO: 42 - protein, 13 aa, Artificial Sequence
  Description of Artificial Sequence Synthetic peptide
AKFVAAWTLKAAA

SEQ ID NO: 43 - protein, 18 aa, Artificial Sequence
  Description of Artificial Sequence Synthetic peptide
GGSGGSGGSGGSGGSGGS